(19) United States
(12) Patent Application Publication
(10) Pub. No.: US 2011/0102177 A1
(43) Pub. Date: May 5, 2011

(54) Diver Monitoring and Communication System

(75) Inventor: Peter Lenard Johnson, Auckland (NZ)

(73) Assignee: Prink Limited, Auckland (NZ)

(21) Appl. No.: 12/673,579

(22) PCT Filed: Aug. 15, 2008
(86) PCT No.: PCT/NZ08/00214

§ 371 Date: Mar. 19, 2010

(30) Foreign Application Priority Data
Aug. 15, 2007 (NZ) 560653

(51) Int. Cl. G08B 21/00; G01N 33/00; A61B 5/08; A61B 5/00

(52) U.S. Cl. 340/540; 73/865.1; 600/532; 600/301

(57) ABSTRACT

The present invention relates to a system, method and software for monitoring a diver comprising: a topside monitoring system 13 on a vessel comprising a receiver for receiving data from at least one diver 12, the data being transmitted using an acoustic modem 207 and specifying information relating to the diver 12, wherein the information relating to the diver comprises the depth of diver, a computer for processing the data 101, and a user interface 111 for conveying the information and the elapsed time of dive to a dive supervisor so that the dive supervisor can manage the dive.

### FIELD OF THE INVENTION

The present invention relates to a diver monitoring and communications system, and more specifically, to a diver monitoring and communication system that allows a diver to communicate with and be monitored by a surface supervisor.

### BACKGROUND OF THE INVENTION

For all SCUBA dives and other types of dives, a diver requires a dive plan. This plan, among other things, includes details relating to the length of the dive, and the decompression schedule. The decompression schedule is obtained from a dive table. Dive tables are issued by various entities, and specify at what depths and for how long at each depth a diver should decompress. A large number of dive tables existing, specifying decompression schedules for a range of different dive depths and bottom times.

For a more complex dive, often the divers are managed “topside” from a dive supervisor. Supervisors are common in commercial dives where the dives are long and/or deep and/or involve complex tasks coordinated for one or more divers. The dive supervisor will set the dive plan prior to the dive. The dive supervisor will manage the dive by monitoring and controlling the actions carried out by each of the divers, and ensuring the dive plan is adhered to.

However, dive supervisors have limited information available to them, and the tools they have available to monitor and control the dive are crude and limited. They lack flexibility to adjust to unforeseen events and circumstances.

Dives can sometimes be monitored through information passed via a tether. However, this is not always practicable. For example, in the case of dives with a large number of divers, such as in recreational dives, tethers cannot be used as they will tangle.

### SUMMARY OF THE INVENTION

It is an object of the present invention to provide for improved management of dives.

In one aspect the present invention may be said to consist in a method of monitoring a diver comprising the steps of:
receiving data from at least one diver, the data being transmitted using an acoustic modem and specifying at least one parameter relating to the diver,
conveying the parameter and elapsed time of dive to a dive supervisor so that the dive supervisor can manage the dive, the parameter and elapsed time of dive forming diver information,
wherein the parameter relating to the diver comprises the depth of the diver.

Preferably the method further comprises the step of determining a decompression schedule for the diver and conveying the decompression schedule.

Preferably the step of determining a decompression schedule comprises receiving input specifying one or more decompression schedules or dive tables, and determining a decompression schedule based on the received input.

Preferably the method further comprises the step of determining one or more alternative decompression schedules based on the conveyed diver information, the step being triggered by received or calculated diver information or by input from a dive supervisor

Preferably the method further comprises the step of conveying the alternative decompression schedules.

Preferably the method further comprises the step of receiving input for selecting one of the alternative decompression schedules and conveying the selected alternative decompression schedule.

Preferably the method further comprises the step of selecting one of the alternative decompression schedules and conveying the selected alternative decompression schedule.

Preferably the step of determining one or more alternative decompression schedules comprises selecting one or more dive tables from a set of dive tables, each dive table specifying one or more decompression schedules, wherein the selection is based on some or all of the diver information.

Preferably selecting one or more dive tables based on some or all of the diver information comprises the step of determining which of the set of dive tables specify decompression schedules that can still be adhered to by the diver based on diver information.

Preferably the method further comprises one or more alarms for alerting a dive supervisor of actions that may be required, wherein the alarms are triggered by the relationship of:
diver information to the decompression schedule, and/or
diver information to specified values and/or states.

Preferably receiving input specifying a decompression schedule comprises the steps of:
receiving input indicating one or more dive tables, or decompression schedules,
receiving input indicating at least one parameter of the dive.

Preferably the parameter is one or more of:
bottom depth of dive,
bottom time,
total dive time.

Preferably the method further comprises the step of recording dive information related to the dive, wherein dive information comprises one or more of any dive related parameter, data, information or input received or generated prior to or throughout the dive.

In another aspect the present invention may be said to consist in system for monitoring a diver comprising:
a topside monitoring system on a vessel comprising:
a receiver for receiving data from at least one diver, the data being transmitted using an acoustic modem and specifying information relating to the diver, wherein the information relating to the diver comprises the depth of diver,
a computer for processing the data, and
a user interface for conveying the information and the elapsed time of dive to a dive supervisor so that the dive supervisor can manage the dive.

Preferably the system further comprises:
one or more sensors on the diver, the sensors for obtaining data relating to the depth of the diver, and
an acoustic modem on the diver for transmitting obtained data to the topside monitoring system

Preferably the computer is adapted to determine a decompression schedule for the diver and convey the decompression schedule via the user interface.

Preferably to determine a decompression schedule the computer is adapted to receive input specifying one or more decompression schedules or dive tables, and determine a decompression schedule based on the received input.

Preferably the computer is further adapted to determine one or more alternative decompression schedules based on the conveyed diver information, the computer being triggered by received or calculated diver information or by input from a dive supervisor

Preferably the computer is adapted to convey the alternative decompression schedules via the user interface.

Preferably the computer is adapted to receive input for selecting one of the alternative decompression schedules and to convey the selected alternative decompression schedule.

Preferably the computer is adapted to select one of the alternative decompression schedules and to convey the selected alternative decompression schedule.

Preferably to determine one or more alternative decompression schedules the computer selects one or more dive tables from a set of dive tables, each dive table specifying one or more decompression schedules, wherein the selection is based on some or all of the diver information.

Preferably to select one or more dive tables based on some or all of the diver information the computer is adapted to determine which of the set of dive tables specify decompression schedules that can still be adhered to by the diver based on diver information.

Preferably the computer is configured with one or more alarms for alerting a dive supervisor of actions that may be required, wherein the alarms are triggered by the relationship of:
diver information to the decompression schedule, and/or
diver information to specified values and/or states.

Preferably receiving input to select a decompression schedule comprises:
receiving input indicating one or more dive tables, or decompression schedules,
receiving input indicating at least one parameter of the dive.

Preferably the parameter is one or more of:
bottom depth of dive,
bottom time,
total dive time.

Preferably the computer is adapted to record dive information related to the dive, wherein dive information comprises one or more of any dive related parameter, data, information or input received or generated prior to or throughout the dive.

In another aspect the present invention may be said to consist in a system for monitoring a diver comprising
one or more sensors on a diver, the sensors for obtaining data relating to the depth of the diver, and
an acoustic modem on the diver for transmitting obtained data to a topside monitoring system.

Preferably the system comprises one or more further sensors on the diver, the sensors for obtaining data relating to one or more of the following parameters:
surface supply breathing gas pressure at surface,
surface supply breathing gas composition at surface (oxygen concentration, nitrogen concentration, helium concentration, carbon dioxide concentration, carbon monoxide concentration and any other gas that is interest to the dive supervisor),
surface supply breathing gas pressure at diver,
surface supply breathing gas composition at diver (oxygen concentration, nitrogen concentration, helium concentration, carbon dioxide concentration, carbon monoxide concentration and any other gas that is interest to the dive supervisor or diver),
self-contained breathing gas supply pressure at diver for primary, secondary or tertiary use (compressed air for breathing, singular or mixed gas for breathing, gas for buoyancy compensation or driving of underwater tools),
self-contained breathing gas composition at diver (oxygen concentration, nitrogen concentration, helium concentration, carbon dioxide concentration, carbon monoxide concentration and any other gas that is interest to the dive supervisor or diver),
dive parameter sensors for altitude, depth, air temperature, water temperature, time.
photographic/video sensors for visual, enhanced visual, IR, UV,
water quality sensors for turbidity, suspended solids, salinity, biological, radioactive parameters,
environmental sensors for temperature, fluid movement and particle movement.
situational awareness sensors, sonar, radar, Doppler tracking of fluid and particles
positional sensors for absolute position and relative position
diver inhaled and exhaled fluid composition (oxygen gas concentration, carbon dioxide gas composition or any other gas parameter of interest to the diver and or diver supervisor)
diver biophysical condition sensors (ECG, EEG, respiration rate, core body and/or skin temperature)
dive supervisor electronic hardware sensors (battery condition, malfunction, self dialogistic)
diver electronic hardware sensors (battery condition, malfunction, self diagnostics)
diver microphone, speaker and microprocessor set for converting analogue sound to digital sound representation for data transmission.
dive supervisor microphone, speaker and microprocessor set for converting analogue sound to digital sound representation for data transmission.

Preferably the diver information can further comprise one or more of the following parameters:
surface supply breathing gas pressure at surface,
surface supply breathing gas composition at surface (oxygen concentration, nitrogen concentration, helium concentration, carbon dioxide concentration, carbon monoxide concentration and any other gas that is interest to the dive supervisor),
surface supply breathing gas pressure at diver,
surface supply breathing gas composition at diver (oxygen concentration, nitrogen concentration, helium concentration, carbon dioxide concentration, carbon monoxide concentration and any other gas that is interest to the dive supervisor or diver),
self-contained breathing gas supply pressure at diver for primary, secondary or tertiary use (compressed air for breathing, singular or mixed gas for breathing, gas for buoyancy compensation or driving of underwater tools),
self-contained breathing gas composition at diver (oxygen concentration, nitrogen concentration, helium concentration, carbon dioxide concentration, carbon monoxide concentration and any other gas that is interest to the dive supervisor or diver),
dive parameter sensors for altitude, depth, air temperature, water temperature, time.
photographic/video sensors for visual, enhanced visual, IR, UV,
water quality sensors for turbidity, suspended solids, salinity, biological, radioactive parameters,
environmental sensors for temperature, fluid movement and particle movement.
situational awareness sensors, sonar, radar, Doppler tracking of fluid and particles
positional sensors for absolute position and relative position
diver inhaled and exhaled fluid composition (oxygen gas concentration, carbon dioxide gas composition or any other gas parameter of interest to the diver and or diver supervisor)
diver biophysical condition sensors (ECG, EEG, respiration rate, core body and/or skin temperature)
dive supervisor electronic hardware sensors (battery condition, malfunction, self dialogistic)
diver electronic hardware sensors (battery condition, malfunction, self diagnostics)
diver microphone, speaker and microprocessor set for converting analogue sound to digital sound representation for data transmission.
dive supervisor microphone, speaker and microprocessor set for converting analogue sound to digital sound representation for data transmission.

Preferably the one or more alarms can be triggered based on one or more of:
surface supply breathing gas low pressure at surface
surface supply breathing gas low quality at surface
surface supply breathing gas low pressure at diver
surface supply breathing gas low quality at diver
primary and/or secondary self-contained breathing gas (or one of its components) low pressure at diver
primary and/or secondary self-contained breathing gas (or one of its components) low quality at diver
diving parameters:
   decompression commitment approaching (user defined, default 5 minutes),
   decompression commitment reached,
   partial pressure of oxygen in breathing gas mixture (user defined, default 1.4)
   accent to fast warning (user defined, default 18 m/min—could be graphical),
   remaining breathing gas warning on self contained supply (user defined default 15 minutes left),
   remaining breathing gas warning on secondary self contained supply (user defined—drop in pressure by 30 psi)
   maximum planned depth approaching (user defined—default 5 msw of planned depth)
   maximum planned depth reached (user defined—maximum 70 msw)
   minimum water temperature approaching (user defined—default 2° C. of set minimum)
   maximum water temperature approaching (user defined—default 2° C. of set maximum)
   minimum water temperature reached (user defined—default 10° C.)
   maximum water temperature approaching (user defined—default 30° C.)
dive environment
   low visibility for safe diver operations (user defined)
   presence of bacteriological activity (user defined)
   maximum level of radioactivity approaching (user defined)
   maximum level of radioactivity reached (user defined)
   current warning (user defined)
situational awareness warning
   approaching selected target (user defined—default range 5 m)
   selected target reached (user defined—default range 1 m)
   unknown target in vicinity of diver (user defined)
biophysical warning
   minimum core body and/or skin temperature approaching (user defined)
   minimum core body and/or skin temperature reached (user defined)
   maximum core body and/or skin temperature approaching (user defined)
   maximum core body and/or skin temperature reached (user defined)
   ECG anomaly (user defined)
   ECG rate high (user defined)
   ECG rate low (user defined)
   EEG anomaly (user defined)
   inhaled breathing gas quality low (user defined)
   exhaled breathing gas quality low (user defined)

In another aspect the present invention may be said to consist in a diver monitoring and communication system comprising:
a dive supervisor monitoring system comprising:
   a micro controller for controlling the system;
   a display for displaying information on a monitored person; and
   communication means for communicating between said dive supervisor monitoring system and a at least one diver monitoring system,
a diver monitoring system including:
   a plurality of sensors having sensor outputs;
   a micro controller for monitoring a plurality of sensors; and
   communication means for communicating the sensor outputs with said dive supervisor monitoring system.

In another aspect the present invention may be said to consist in a diver monitoring system comprising:
a plurality of sensors having sensor outputs;
a micro controller for monitoring a plurality of sensors; and
communication means for communicating the sensor outputs with said dive supervisor monitoring system.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

As used herein the term “and/or” means “and” or “or”, or both.

As used herein “(s)” following a noun means the plural and/or singular forms of the noun. The entire disclosures of all applications, patents and publications, cited above and below, if any, are hereby incorporated by reference.

In this specification, where reference has been made to external sources of information, including patent specifications and other documents, this is generally for the purpose of providing a context for discussing the features of the present invention. Unless stated otherwise, reference to such sources of information is not to be construed, in any jurisdiction, as an admission that such sources of information are prior art or form part of the common general knowledge in the art.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting

### BRIEF DESCRIPTION OF THE DRAWINGS

Disclosed embodiments and methods of utilizing the invention will be further described, with reference to the accompanying figures, by way of example only and without intending to be limiting, wherein;

FIG. 1 is a block diagram of a system according to one embodiment of the invention,

FIG. 2a is a block diagram of a topside monitoring system,

FIG. 2b is a block diagram of software utilised by the topside monitoring system,

FIG. 3 is a block diagram of a diver-side system,

FIG. 4 shows a main screen of a user interface of the system,

FIG. 5 shows a monitor screen of the system,

FIGS. 6a to 6c shows an adjustable dive table provided by the system,

FIG. 7a shows a selected dive table for a dive plan,

FIG. 7b shows a monitor screen of the system showing a decompression schedule according to the dive table in FIG. 7a,

FIGS. 8a and 8b show a process for altering a dive plan,

FIGS. 9a-9d show alternative dive tables for an alternative dive plan,

FIGS. 10a and 10b show user login and creation screens of the system,

FIGS. 11-13 show diver registration screens of the system,

FIG. 14 shows a setting screen of the system,

FIGS. 15-18 show alarm setting screens of the system,

FIG. 19 shows an input screen for dive planning,

FIG. 20 shows an input screen for weather information,

FIGS. 21 and 22 show alternative embodiments of the top-side and diver-side systems,

FIG. 23 shows the range of the acoustic modem, and

FIG. 24 shows an alternative monitor screen.

### DETAILED DESCRIPTION OF THE INVENTION

### Overview of System, Method and Software

The present invention provides a system 10 and method to enable a dive supervisor positioned topside in a vessel 11 (or shore based location) to manage one or more underwater divers 12. The supervisor monitors and controls each diver by advising what actions they should take. This comprises advising them as to when and how to implement their decompression schedule.

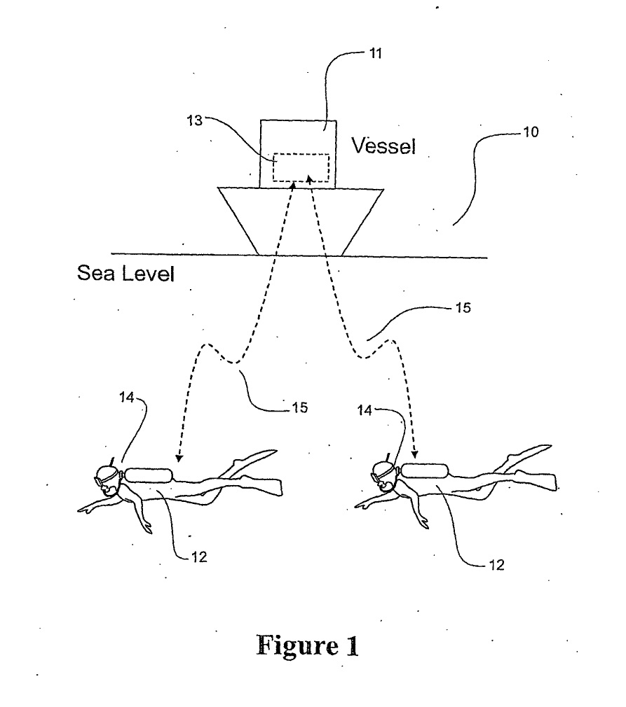

FIG. 1 shows a broad overview of a preferred system 10 according to the invention. The system comprises a topside monitoring system 13 and a diver-side system 14 worn by the diver at various locations on their body. The topside monitoring system 13 is disposed on the vessel 11. Typically this will be the vessel 11 from which the divers 12 conduct the dives, but this is not essential. The topside system comprises communication means and computer systems to enable the dive supervisor to receive information relating to the divers 12 and to process and act on that information. The diver-side system 14 generally comprises sensors for obtaining information relating to the diver and the dive, computer systems for processing the information and or conveying it to the diver, and a communications system for communicating information topside.

The topside monitoring system 13 and the diver-side system 14 are in communication 15 via acoustic modems/transducers forming acoustic transceivers (typically referred to as “acoustic modems”). The acoustic modems comprise part of the topside 13 and diver-side systems 14. The information from a diver 12 is transmitted via the acoustic modem to a receiving unit on the surface which forms part of the topside monitoring system. The monitoring system 14 records, calculates and displays various parameters. The monitoring system 13 allows a dive supervisor to closely track each diver's status. Then, using the system via voice or data communications, the diver can be given instructions on aspects related to the dive, including decompression commitment.

In parts of the present specification, the preferred embodiment will be described in relation to one diver. It will be appreciated that the system and method described could be configured for a number of divers. Each diver would wear the diver side system herein described, which would communicate with the topside system.

### Topside Monitoring System

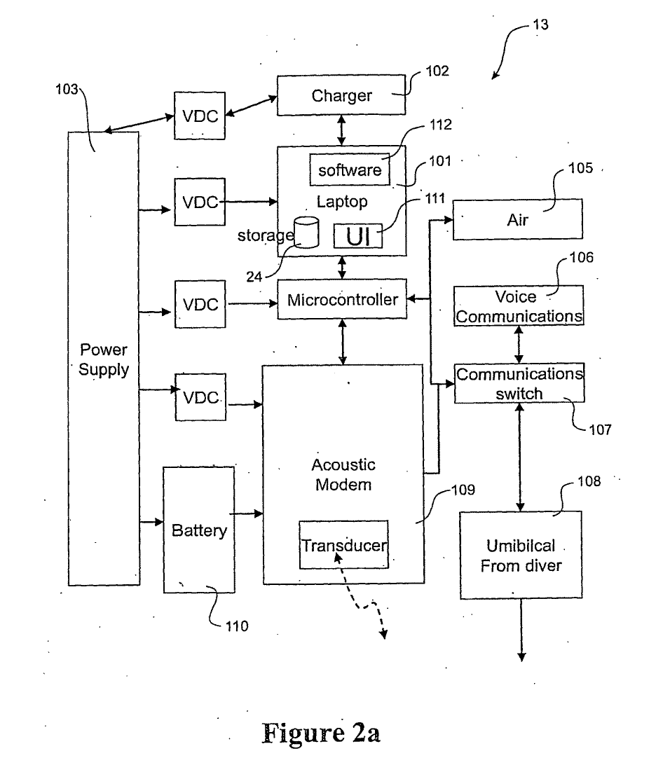

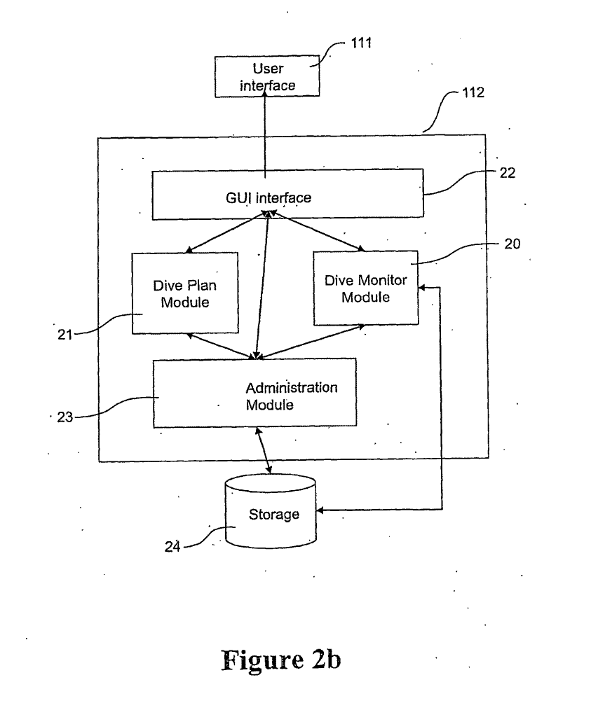

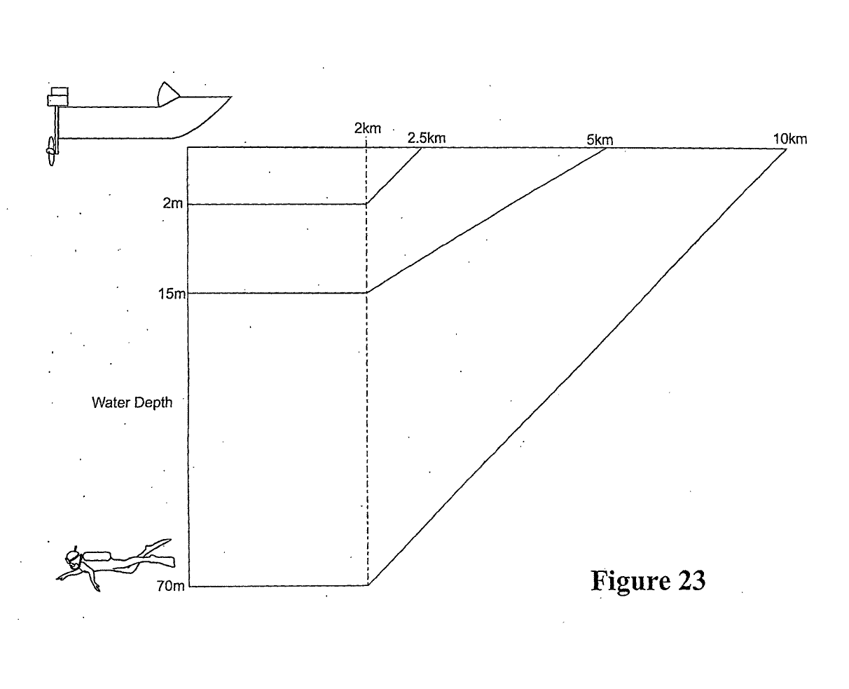

FIG. 2a shows one embodiment of the topside monitoring system 13 in more detail. The topside monitoring system comprises a micro controller 120 for controlling the system. The micro controller is coupled to an acoustic modem/transducer 109 via an data connection. This forms the acoustic modem transceiver for voice and data communications, referred to typically as an “acoustic modem”. Acoustic modems convert electrical signals to tones and back again. In the present invention, the sounds are transmitted through water. Suitable acoustic modems include Link Quest Inc: UWM1000, UWM2000, UWM2200, UWM2000H, Tritech International Limited: AM100, AM300, Teledyne Benthos: ATM 885, ATM887, DSP Com: AquaComm Modem and Wireless Fibre Systems: RAM300, S5510, S1510. Acoustic modems have a working range measured in kilometres. The deeper the modem, the better the range, as shown in FIG. 23.

The acoustic modem 109 of the topside monitoring system 13 receives data obtained by various sensors on the diver 12 that are transmitted via the diver's respective acoustic modem. The acoustic modem 109 can also transmit the data to the diver via the diver's acoustic modem. Similarly, voice communications with the diver can take place via the acoustic modem 109, which will encode voice communications into appropriate acoustic tones for transmission. Any received voice or data received via the transducer will be processed by the acoustic modem and passed to a voice communications device 106, such as a headset. In an optional alternative, there may also be an umbilical or tether 108 between the diver-side system 14 and the topside monitoring system 13. This can carry transmitted and received data/voice. A communications switch 107 passes voice communications received via the modem 109 or tether 108 to the voice communications device.

The micro controller 120 is also connected via an data connection to a computer 101, such as a laptop. The computer 101 comprises a user interface 111 so that the topside system 13 can convey information to a dive supervisor and so that a dive supervisor can interact and control the topside system 13. The user interface 111 preferably takes the form of a display screen, keyboard, mouse and the like. The computer executes software 112 that carries out various functionalities of the monitoring system 10, including user interface functionalities, processing and data acquisition/transmission. The software is shown further in FIG. 2b

The acoustic modem 109 and laptop 101 both have battery packs and/or chargers 110, 102, which are connected to a suitable power supply unit 103. The power supply unit provides various voltages as required.

### Diver-Side Monitoring System

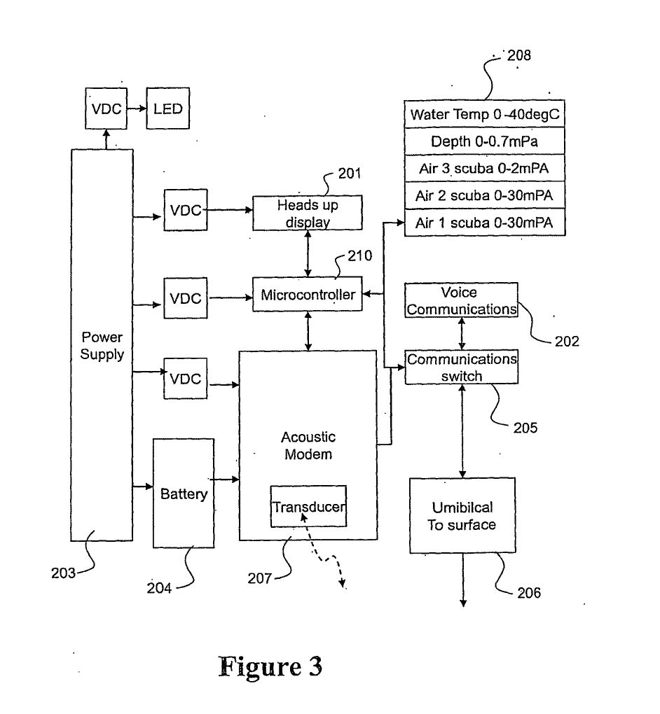

The diver-side system 14 is shown in FIG. 3. The components of this system will be worn on various points on the diver's 12 body. The diver will have a number of sensors 208 arranged to obtain data on a number of parameters relating to diver status. These parameters comprise air pressure, water temperature, body temperature, water pressure and the like. Further sensors to monitor stress on the body of a diver can also be incorporated within the system. These might comprise sensors for obtaining physiological data on the diver (ECG/EEG and the like) and/or gasses (inhale/exhale/blood gasses and the like). A list of further possible parameters and sensors is set out in appendix A. FIG. 3 shows a small selection of sensors 208, for illustrative purposes. These sensors capture data on water temperature, diver depth, and pressure of three separate air supplies.

The diver-side system 13 is controlled by a micro controller 210, which is coupled to the sensors 208. The micro controller 210 processes the various parameters obtained by the sensors 208 and/or displays them or otherwise conveys them to the diver. Preferably, the micro controller conveys the information to the diver via a heads up display 201 that is coupled to the micro controller 219 via an data interface. Other means of conveying the information to the diver 12 could be used, such as a wrist mounted dive computer or display.

The micro controller 210 is also coupled via an data interface to an acoustic modem/transducer 207 (“acoustic modem”) for emitting and receiving data/voice in the form of acoustic signals. The data received via the sensors 208 can be transmitted to the topside monitoring system via the acoustic modem 207. The acoustic modem 207 can also receive data and voice communications transmitted from the topside monitoring system 13 via its acoustic modem 109. Any data that is received can be processed by the micro controller 210 and conveyed to the diver via the heads up display 201 or other suitable means, where appropriate. Any voice communications can be conveyed via an appropriate voice communications headset or similar 202. Where, optionally, a diver is coupled to the topside monitoring system 13 via an umbilical or tether 206, any data or voice communications can be relayed to the micro controller and conveyed by the heads up display 201 or the voice communications head set 202 as required. A communications switch 205 will switch data/voice communications received from the transducer or the tether 206.

The micro controller 210 is powered by a power supply unit 203 and a battery pack 204 that provide the required DC voltages to operate.

### Overview of Functionality of the System and Software and Methods for its Operation

A dive monitoring module 20 is the main operational module of the software 112 in the topside monitoring system 13. There is also a dive plan module 21. Within these modules 20, 21 the dive is planned, documented, initiated, monitored and/or recorded. The user interface 111 on the topside monitoring system computer 101 is controlled to periodically or continuously display the dive status of the diver. This status is based on data received from the sensors 208 and other information. The data received from the sensors specifies parameters of the diver/dive. Other parameters, such as elapsed time, can be generated. Together, these parameters form dive or diver information, and convey dive/diver status. For each monitored diver, the dive monitor module 20 will display (via the user interface 22, 111):
the time from when the diver left surface (elapsed dive time),
the diver's bottom time left,
diver depth,
decompression commitment,
water temp near diver (and/or elsewhere),
surface air supply pressure at the surface,
surface air supply pressure at the diver,
primary bottled air supply pressure,
secondary bottled air supply pressure (this will be the bailout bottle if on surface supply or primary bottled air expires),
air remaining warning,
diver recall.

This list is not exhaustive. Any information that is obtained from the diver or elsewhere could be displayed. Not all of the above information needs to be displayed. Only the most pertinent information might be displayed, for example.

Before beginning a dive, checks can be made for data communications. Once these tests are complete the system will start recording the dive once the diver hits the water (using a conductivity link and seawater immersion) or when the diver reaches a nominated depth of 1.0 m or more. The dive supervisor will be able to select the type of dive being undertaken.

The dive monitoring module 20 in the software 112 allows a dive supervisor to select the type of dive being undertaken and the supply of air available. The selection choice will include: dive on surface supply, primary and bail out bottle dive, primary bottle only dive (default), diver dependant—some may be on SSA others on SCUBA.

The display unit is adjustable “on the fly” for brightness.

The software 112 on the topside monitoring system 13 comprises an administration module 23 allows the dive supervisor to set the units for each parameter. This will determine how the units are displayed. The default units (and all the units used for calculation are to be in metric units. The displaying of other units will require the units to be converted just prior to display and/or calculations.

For each diver 12 being supervised, details are stored (in storage facility 24) by the topside monitoring system 13, these include; name, date of birth, diver qualifications, divers dive log (software generated and or manually entered) and a diver identifier. For each diver it is possible to view all information, print out as required and attach documents to each diver.

The topside monitoring system can also automatically update the diver log with every dive that is monitored with the system.

The modem will optionally allow other divers to communicate with and view data on dive buddies.

Optionally the overall system might allow a photo link between the diver and the surface. If a photo link is used the modem may need to be upgraded for greater data capacity, however the diver is able to take photos that are instantly transmitted to the surface.

Before using the system, the dive supervisor logs into the system 13 using the administration module 23. Prior to that, they set themselves up as a user. A diver is registered on the topside monitoring system 13 using the administration module 23 before a dive can be managed for that diver. Registering comprises inputting information relating to the diver, which will comprise at least the diver's name or other label. It is envisaged that the diver's name/label could be entered on the fly from the monitoring screen.

The software 112 of topside monitoring system 13 comprises a dive planning module 21 that allows a diver and a dive supervisor to plan a dive and revise the dive plan as necessary. This comprise the prediction of the decompression commitment of the diver using received dive status parameters and the DCIEM or other recognised dive tables. The dive planning module can adjust a dive plan, for example, when alternative gasses such as Nitrox are used. For such gasses the “equivalent air depth” method with mixture checks for partial pressure of Oxygen and oxygen toxicity exposure duration can be used. From the dive duration an estimate of the amount of air needed for the dive will be calculated. The estimation can be a general estimation or an estimation for the specific diver, depending on whether there are sufficient dives logged for the diver within the system. The dive plan can be printed out to a printer from within this module.

### Detailed Description of Topside Monitoring System Functionality and its Use.

### Dive Monitoring

The topside monitoring system 13 provides an application (software) 112 and graphical user interface 22, 111 that allows the dive supervisor to monitor the various parameters and other aspects of the dive, and as a result make decisions on how to direct the divers.

Typically, the role of the dive supervisor is to manage each of the divers and direct what actions they take. Prior to commencing a dive, the dive supervisor will prepare a dive plan, which comprises among other things, the decompression schedule. A decompression schedule indicates how a diver should decompress—namely during the ascent, what depths they should stop at, and for what length of time. Dive tables specify decompression schedules for various bottom depths, bottom times, and any other relevant parameters. Of key importance is ensuring that divers keep to the decompression schedules. The dive supervisor will plan the tasks that are to be completed during the dive. From the depth, time taken and available air, the supervisor must determine how best they can achieve the tasks while still keeping to the recommended decompression schedules as specified by appropriate dive tables.

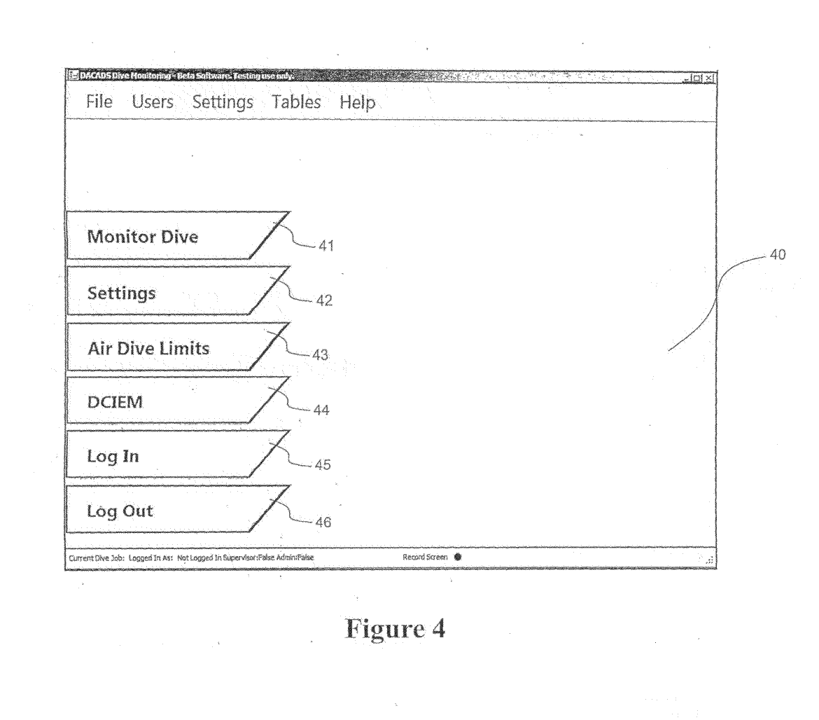

FIG. 4 shows the main screen 40 of the topside dive monitoring system user interface 111. The screen enables a dive supervisor to access log in 45 and log out 46 screens, and access adjust settings 42 screens. The screen also allows the diver to access air dive limits 43 and DCIEM dive tables 44. The air dive limit table relates to the table in the DCIEM manual. The fact we are using DCIEM tables mean that we will be adhering to the air dive limits. This table assesses the oxygen toxicity at depth. The screen also provides access to the monitor dive module 21, 20 main screen for monitoring and planning dives.

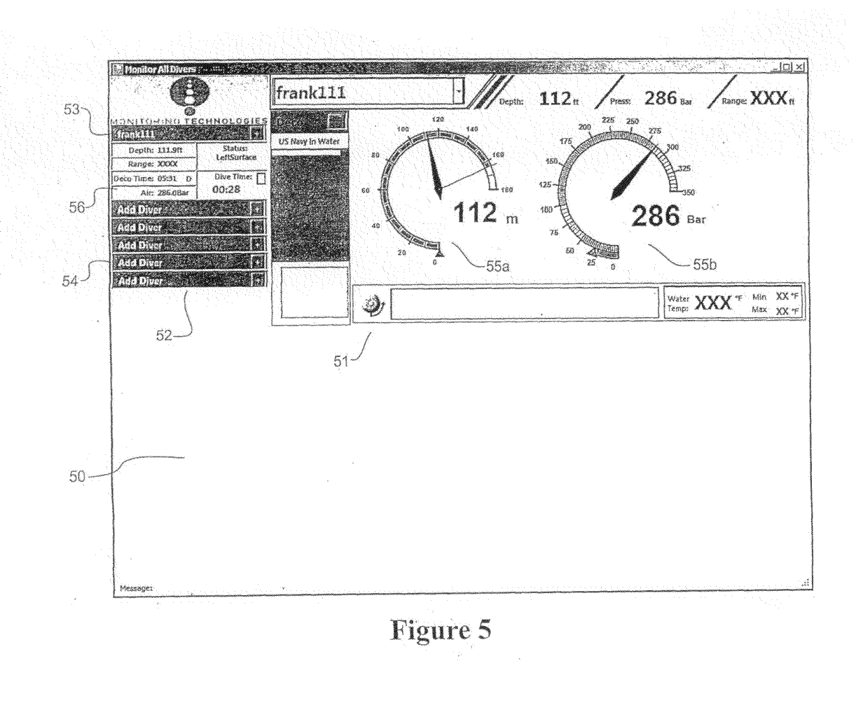

Once a dive supervisor has logged into the system, they can prepare a dive plan. Next, they access the dive monitor module 20, and a screen 50 will be displayed as shown in FIG. 5. This is the main control panel 51 that enables the dive supervisor to monitor the various parameters of the dive, including those parameters that are being received (via sensors) from the one or more divers on the dive. Further, it allows for various actions to adjust the dive plan.

On the left hand side of the screen 51, each of the divers participating in the dive are shown 52. Each diver is registered on the system, and the registration comprises information pertaining to the diver that is important to plan and control their dive. In FIG. 5, one diver (who has already been registered) has been named for this dive—“Frank 111” 53. Additional divers 54 can be added to the dive by clicking an icon and selecting a registered diver. This control panel allows the dive supervisor to therefore monitor multiple divers at once.

In FIG. 5, the screen shows the following information (parameters) 56 for diver Frank 111
current depth (111.9 feet),
range from the topside vessel (100 ft),
the deco time—being time left until the diver must decompress (5:31),
remaining air (286.0 Bar),
their elapsed dive time (00:28), and
status (left surface).

The diver depth and available air is also shown in dial form or graphical form 55a, 55b.

As data is captured from the various parameter sensors 208 on the diver 12, the data will be transmitted via the acoustic modem 207 to the topside monitoring system 13 and displayed as required to the dive supervisor via the screen 50. The dive supervisor can monitor the various parameters 56 and advise the diver on what actions to take through voice, text message or other suitable communications. Only a small subset of the possible parameters 56 are shown on the screen in this example. It will be appreciated that other further parameters could be displayed as and when necessary. Parameters such as time remaining at current depth, water temperature and bottom time could also be shown, among others. In one embodiment, the acoustic modem on the diver-side system will update the data regularly, such as at least every 3-4 seconds.

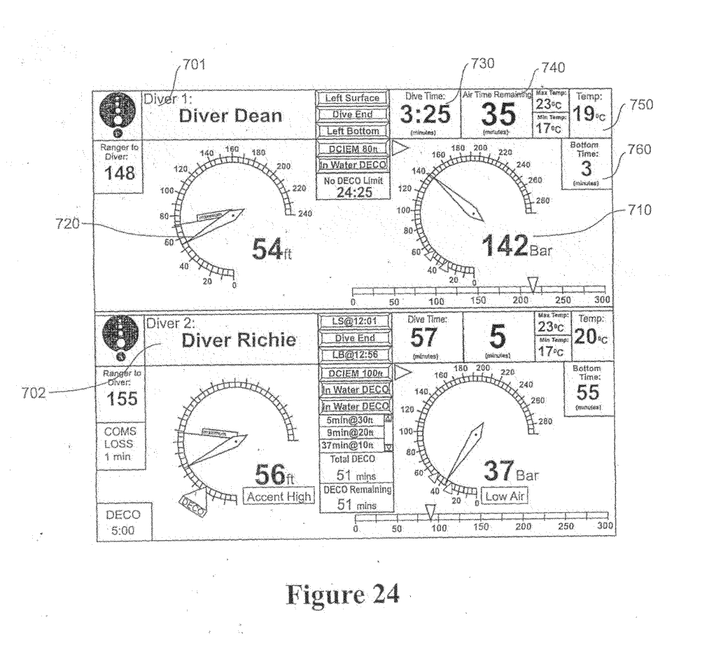

An alternative, more detailed dive screen in an alternative format is shown by way of example in FIG. 24. Here, the information for two divers is illustrated. For each diver 701, 702 the supervisor will be able to see the remaining air pressure 710, the current depth 720, the diver name 701, the dive time 770, the time remaining at the current depth 740, the water temperature 750 and the bottom time 760.

To assist the dive supervisor manage the dive, alarms are provided. These help warn the supervisor of changes in circumstances that require action, emergency situations, or problems with decompression adherence. The incoming data from the diver and other parameters are processed by the software, and alarms will be triggered if an alarm condition is met for any of these parameters. A large number of alarms could be configured for a large range of parameters or combination of parameters. As an example, alarms can be triggered based on:
surface air supply pressure low at surface (user defined, default 140 psi),
surface air supply pressure low at diver (user defined, default 120 psi),
primary bottle air supply pressure low (user defined, default 50 bar),
secondary bottle air supply pressure drop (user defined, default more than 5 bar in 3 minutes),
secondary bottled air supply pressure low (user defined, default 50 bar),
decompression commitment approaching (user defined, default 5 minutes),
decompression commitment reached,
ascent too fast (user defined, default 18 m/min),
remaining air low (15 minutes left on primary bottled air supply),
remaining air low (15 minutes left on secondary bottled air supply),
ascent too rapid (user defined, default 18 m/min +/−3 m/min),
air use warning (user defined, default 15 minutes once decompression commences),
physiological data problems, comprising ECG, EEG, etc, respiration rate,

The alarm will be provide via the user interface through audible or visual cues. A larger (although not exhaustive) list of alarms is set out in Appendix B.

A system parameter display module can also display and allow a user to print out various system parameters including various dive tables. A dive log can additionally be printed out for each diver.

Using these tools, the dive supervisor can determine what actions need taking, and advise the diver appropriately and/or plan/action an intervention on the dive (such as sending a rescue driver to retrieve the diver)

### Dive Planning and Decompression Schedule Monitoring

Prior to sending a diver on a dive, the dive supervisor will plan the dive. A key part of the dive planning involves determining the dive profile, complete with bottom time and scheduled decompression stops. To do so, the dive supervisor will select an appropriate dive table from the store of dive tables available in the topside dive monitoring system. These will be standard dive tables provided, for example by US Navy, DCIEM and Buhlmann.

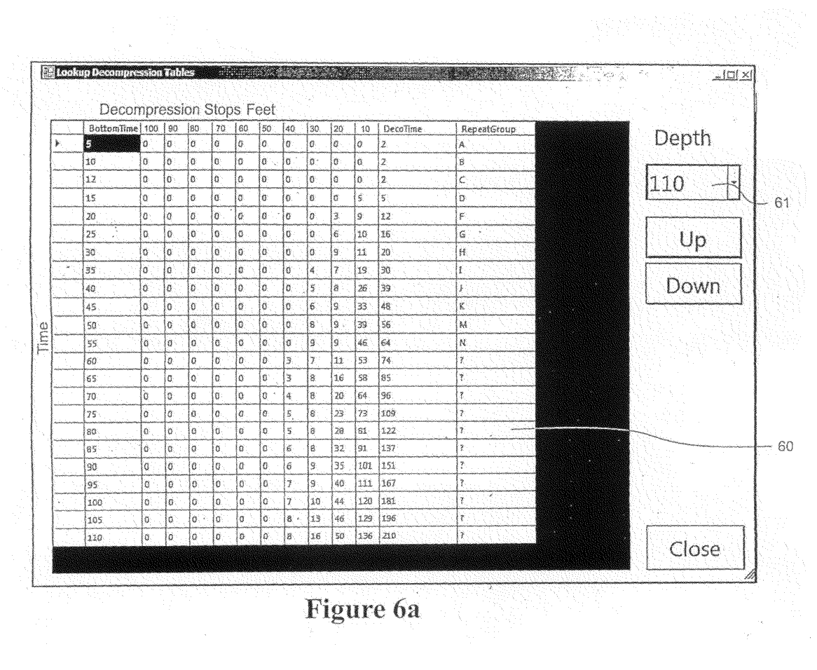

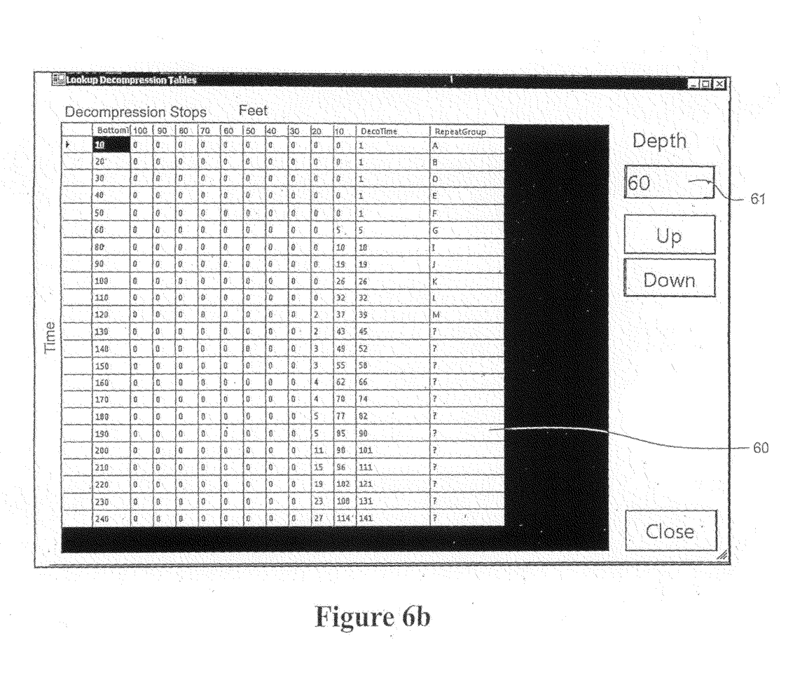

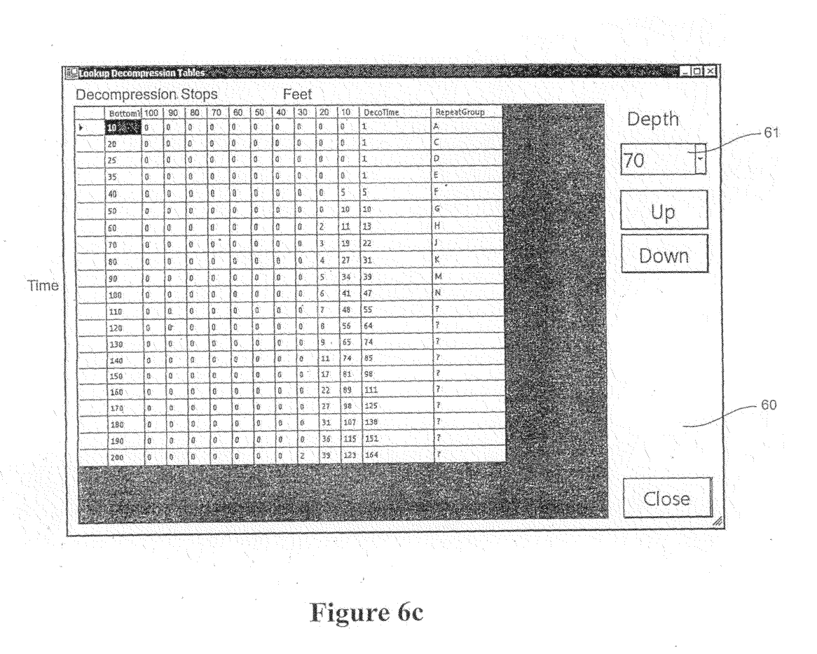

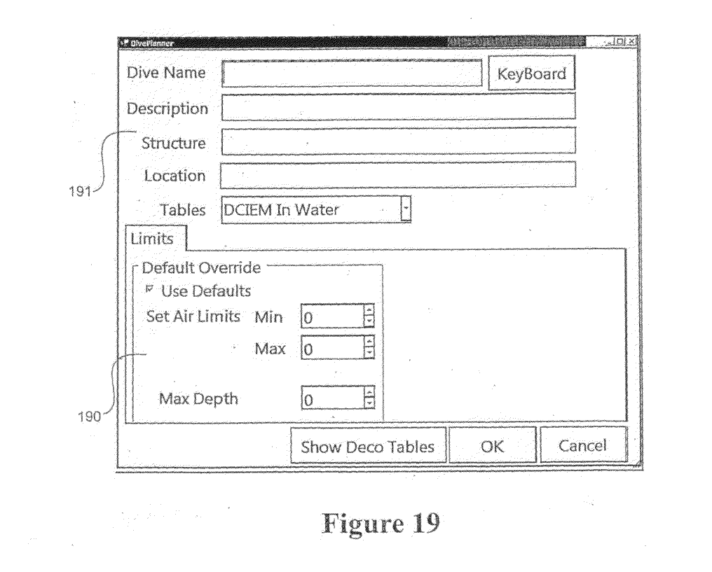

A generic dive table 60 is shown in FIG. 6a, by way of example. This can be accessed from a dive planning screen (see e.g. FIG. 19) in the dive plan module 21. From a drop down box 61, the supervisor can select the bottom depth that is intended for the dive. In this case the bottom depth is 110 feet. Once the bottom depth has been specified, a dive table 60 is shown that specifies the required decompression schedule for dives with bottom times of various lengths. The decompression stops both in time and depth based on the time spent at the bottom depth are shown. In this case, from left to right, each decompressions stop relates to 10 feet of depth. As and when the bottom depth is re-selected in the drop down box, the displayed dive table will be recalculated and re-displayed, showing the different decompression stops, as appropriate. Tables for different depth selections for 60 and 70 feet are shown by way of example in FIGS. 6b, 6c.

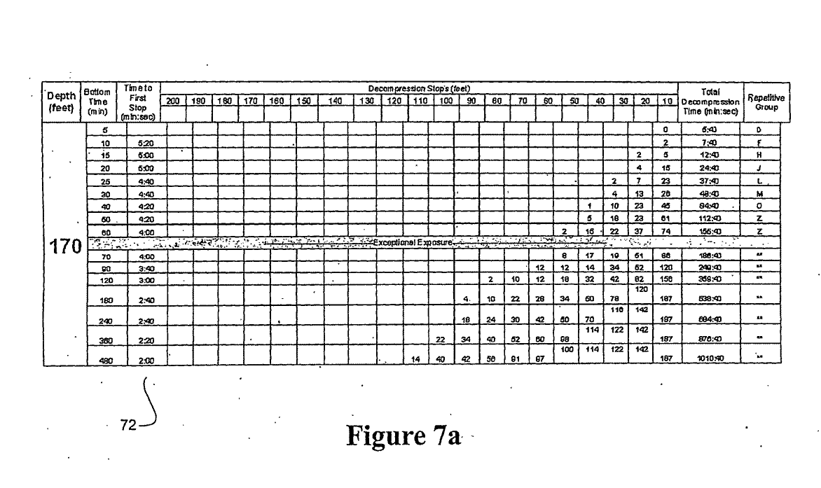

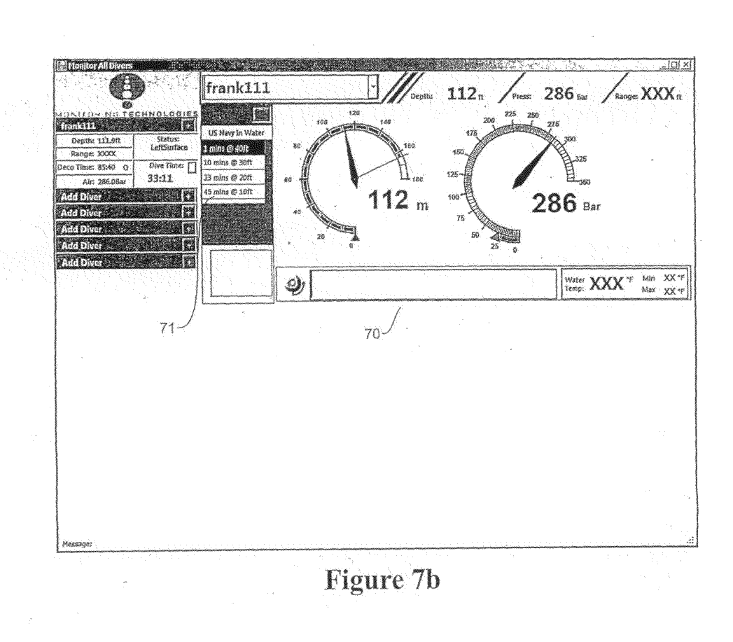

When the depth has been specified by the dive supervisor, the dive will commence on the basis of the decompression schedule according to the selected dive table and the bottom time (in this case 33 minutes). FIG. 7a shows a selected dive table 72 for 170 feet with a bottom time of 33 minutes. FIG. 7b shows the dive monitoring screen 70 during a dive. In this case, Frank 111 has been diving for 33 minutes and 11 seconds and his depth and air supply are displayed. The dive has proceeded on the basis of the dive chart 72 selected by the dive supervisor prior to the dive commencing. In this case, the dive supervisor selected the chart in FIG. 7a which is based on the bottom depth being 170 feet. In the decompression window 71, the topside monitoring system 13 has calculated or determined (from the selected dive table in FIG. 7a) the various stops and times for decompression. These are displayed as a decompression schedule in the decompression window 71. The schedule specifies that the diver will need to decompress for:
1 minute at 40 feet,
10 minutes at 30 feet,
23 minutes at 20 feet, and
45 minutes at 10 feet.

As the dive progresses, the dive supervisor will communicate with the diver to indicate when they should ascend to the next decompression depth, and how long they should remain there. Information will continually be communicated from the diver to the topside monitoring system 13. The topside monitoring system 13 will look at the depth and time of the diver, and compare this to the decompression schedule. If the diver is not adhering to that schedule, appropriate alarms will be conveyed to the supervisor and an omitted decompression schedule, as per the dive tables, will be calculated and displayed to the dive supervisor.

For example, the dive monitoring system will detect when the diver has reached 40 feet, being the first decompression stop. If the diver does not spend at least 1 minute at this depth, namely the dive system notices that the diver ascends beyond that depth sooner than 1 minute, then the system will provide an alarm. A similar event will occur for the other decompression stops. The alarm alerts the dive supervisor to the issue, in case they need to take action. Further, the information will be recorded for later analysis if necessary. If a decompression schedule is not adhered to, the system may also sound alarms or create lock outs for future dives for that diver.

### Dive Plan Adjustment

Traditionally, prior to a dive for a particular diver, a dive supervisor would select the required dive table that is appropriate for the diver and tasks at hand. Throughout the dive they would ensure that the diver is keeping to the decompression schedule. However, if unforeseen circumstances occur, the selected dive table may no longer be suitable, or it may not actually be possible to adhere to the dive schedule specified by the dive table. There is no flexibility for the dive supervisor to readjust the dive plan. This can result in loss of productivity, and worse can endanger the diver's safety.

The functionality of the topside monitoring system and software enables the dive supervisor to prepare a dive plan and then revise the dive plan throughout the dive, as necessary, based on unforeseen events. This can be done by way of a combination of the data received from the diver and elsewhere, processing done by the topside monitoring system and experience/knowledge of the supervisor. Together this information along with functionality of the topside monitoring system allows the dive supervisor to alter the dive plan in an appropriate manner. This enables more productive dives and also improves diver safety.

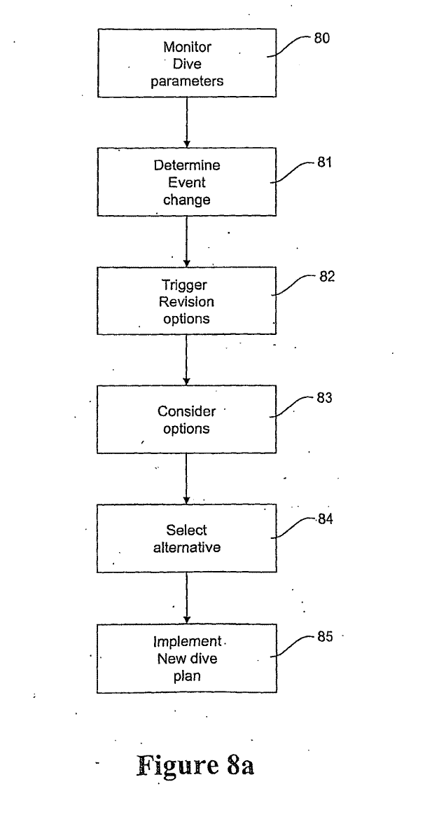

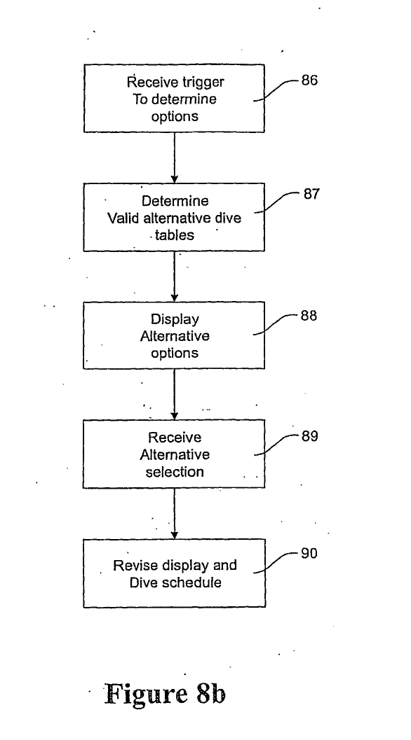

The process as conducted by the dive supervisor and the topside monitoring system is shown in FIGS. 8a and 8b. Referring to FIG. 8a, the dive supervisor pre-plans the dive, which comprises selecting a decompression schedule in the usual manner, such as shown in respect of FIG. 6a. The dive supervisor then sends the diver and monitors the dive parameters in the usual way, step 80, using a screen such as shown in FIG. 7b.

If the dive supervisor determines some significant change in circumstances, step 81, from the received dive parameters that requires an adjustment of the dive plan, they will then operate the monitoring system via the user interface to indicate that a new dive plan is required, step 82. The topside monitoring system will then convey alternative decompression schedules, which can be reviewed by the dive supervisor, step 83. Based on experience, the parameters and their understanding of the situation, the dive supervisor will select a suitable alternative decompression schedule, step 84. Based on the selection, the topside monitoring system will update the decompression schedule and convey this to the dive supervisor. The dive supervisor will then continue management of the dive in accordance with the new decompression schedule, step 85.

In this process, the topside monitoring system operates in the manner depicted in FIG. 8b. The system conveys a decompression schedule according to the original dive plan, along with parameters received from the diver. At some point it may receive input from a dive supervisor via the user interface indicating that a revised dive plan is required, step 86. The topside monitoring system will then run an algorithm to parse the available dive tables, and filter out those that are not appropriate or not possible under the current diver status, step 87. The algorithm will comprise the computer utilising the various diver parameters such as the available air, bottom time, bottom depth, elapsed time and any other suitable parameters, to determine which of the dive tables specify a decompression schedule that can still be adhered to by the diver based on their current situation. The dive tables that still can be adhered to will be conveyed to the dive supervisor via the user interface, step 88. The topside monitoring system will then receive input from the dive supervisor indicating which of the alternative dive tables should be used, step 89. Upon receiving a selection, the topside monitoring system will update the decompression schedule according to the new dive table and convey this through the main user interface to the dive supervisor, step 90. Any alarms will be triggered based on the new decompression schedule.

### Dive Plan Adjustment—Example Scenario

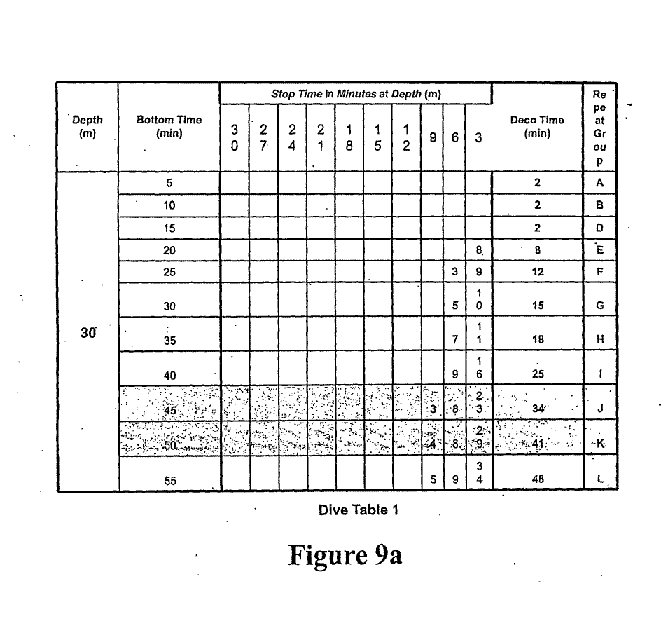

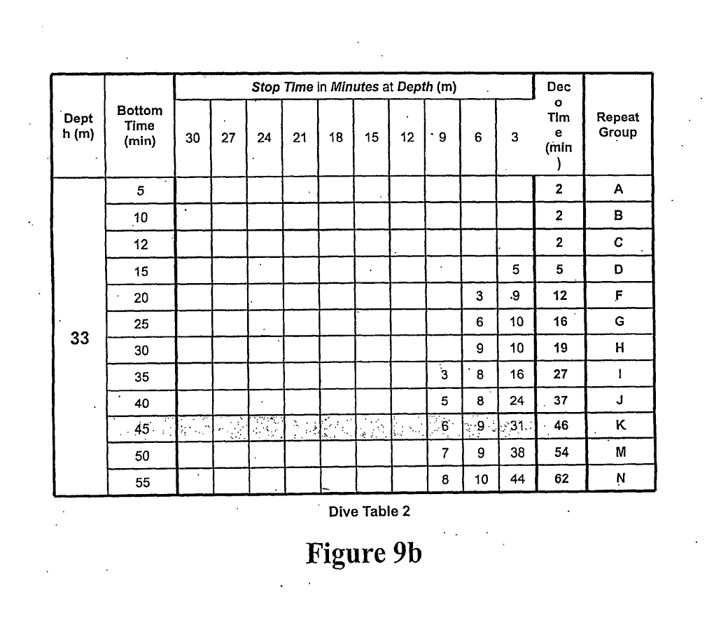

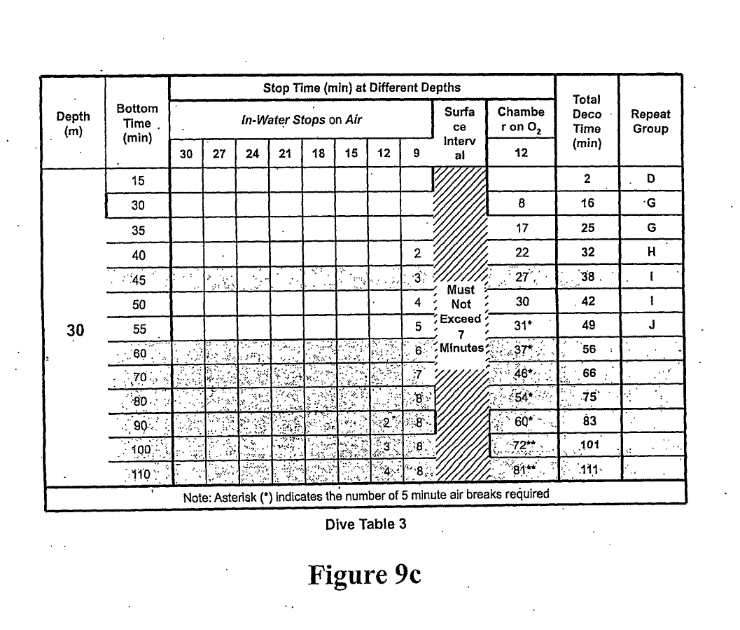

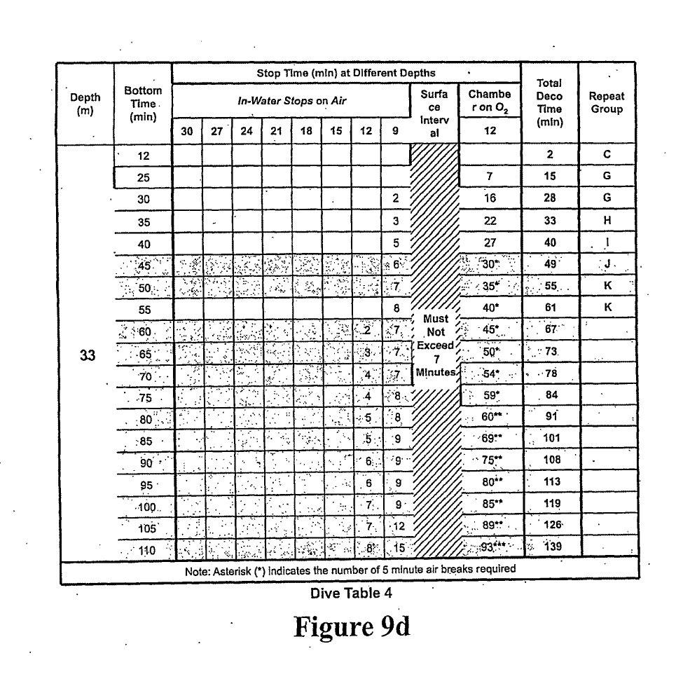

An example of a revised dive plan scenario is described with reference to FIGS. 9a to 9d. Using the monitor system via the user interface, the dive supervisor initially determines a dive plan for a diver. The dive plan specifies the intention to descend to a depth of 30 metres with a bottom time of 45 minutes. Using the interface 111, 22, a dive table is selected from the available tables by selecting the bottom depth of 30 metres. In this case, DCIEM 30 metre table is selected, as shown in FIG. 9a (dive table 1). This table specifies standard “in water” decompression commitment stops of:
1. 3 minutes at 9 m,
2. 8 minutes at 6 m, and
3. 23 minutes at 3 m

For a dive at 30 m with a bottom time of 45 minutes.

The dive is commenced as per the plan with the diver descending to the 30 metre bottom depth. At arrival at that depth, the topside monitoring system 13 receives data from the diver 12 indicating the water temperature and it records a water temperature at the bottom of only 6° C. This low temperature was unexpected.

At this stage, only 6 minutes of the planned 45 minute dive has passed, but given the low temperature, the dive supervisor then considers a more conservative decompression routine would be appropriate for the diver to compensate for the colder water temperature. Using traditional methods, trying to adjust for this would be ad hoc, prone to errors and potentially dangerous. However, the topside monitoring system of the present invention assists the modification of the dive plan, and in particular the decompression schedule, for this unexpected change in circumstances.

Upon the dive supervisor indicating via the user interface 111, 22 that they want to change the dive plan, the topside system 13 then determines and presents various options to the supervisor taking into account the recorded constraints. At this point in the scenario the constraints are:
1. Bottom time must be more than 6 minutes (as 6 minutes has already passed).
2. Minimum diver depth must be 30 m (as the diver has already reached 30 m).

The dive supervisor also makes the decision that the dive task will continue and the diver will be on task for the full planned 45 minutes.

Based on this set of information, the monitoring system 13 can then run/offer various scenarios to the dive supervisor for alternative decompression schedules. The monitoring system 13 does this by using its software 112 to compare the desired bottom time, elapsed dive time and bottom depth to the available dive tables, and selecting those that are still available/appropriate based on these parameters. Some will be excluded as not appropriate. For example, if the dive time is 45 minutes, any dive tables that require a decompression schedule of over 45 minutes bottom time will not be appropriate. As another example, any dive tables that correspond to depths of less than 30 metres will also not be appropriate, as the diver is currently already at 30 metres. The monitoring system software 112 can be programmed with rules to eliminate inappropriate dive tables based on the current parameters of bottom time, elapsed time, bottom depth, current depth, available air, and any other relevant parameters. Any dive table that has a decompression schedule that cannot be adhered to based on the current information will not be provided by the topside monitoring system 13.

In the current example, the topside monitoring system selected dive tables resulting in the following possible alternative scenarios/decompression schedules.

### Scenario Option #1

Dive on the next table down using the “in water” decompression schedule to compensate for the colder than expected water temperature. That means using the 33 m table, for 45 minutes (see dive table 2, FIG. 9b) giving a decompression schedule of:
a. 6 minutes at 9 m,
b. 9 minutes at 6 m, and
c. 31 minutes at 3 m.

Using this table would increase the total decompression commitment for the diver from 34 minutes to a total of 46 minutes.

Given the exposure potential of the recorded water temperature it may be prudent to look at another option to limit the diver “in water” exposure by switching to a surface oxygen decompression routine.

### Scenario Option #2

Minimise the “in water” exposure and switch from “in-water” decompression to a surface O₂ decompression routine using the 30 m table for surface decompression (see dive table 3, FIG. 9c), giving a schedule of:
a. 3 minutes at 9 m in water,
b. no more than a 7 minute interval on the surface for transfer to a hydrobaric chamber,
c. 27 minutes in the chamber at an equivalent depth of 12 m while on oxygen.

This scenario presents an aggressive decompression strategy, but minimises the “in-water” exposure for the diver. This is beneficial as it would reduce exposure to cold water. With the scenario the in water time is shortened by 31 minutes.

### Scenario #3

Use a more conservative scenario of surface O₂ decompression to limit “in water” decompression time but use a deeper table to compensate for the colder than expected water temperature by switching the dive plan to the 33 metre surface O₂ dive table (see dive table 4, FIG. 9d). This now gives a decompression schedule of:
a. 6 minutes at 9 m in water,
b. no more than a 7 minute interval on the surface for transfer to a hydrobaric chamber.
c. 30 minutes in the chamber at an equivalent depth of 12 m while on Oxygen with one break breathing air for 5 minutes.

This plan limits the “in water” exposure (28 minutes less than original dive plan) for the diver, but gives a level of conservatism to compensate for the lower than expected water temperature.

### Scenario #4

The dive supervisor could decide to stay on the original dive plan knowing that they can switch plans “on the fly”.

In this case the supervisor chooses scenario #4. The dive continues and the diver finishes the task and leaves the bottom after 48 minutes (some 3 minutes over dive plan). At this time, the software is on the 30 m “in water” decompression schedule, which calculates the following decompression schedule (see Dive table 1, FIG. 9a):
a. 4 minutes at 9 m,
b. 8 minutes at 6 m, and
c. 29 minutes at 3 m

But, on the way up the diver might complain about the cold and the diver now has to spend some 41 minutes in subsurface decompression. To remedy this situation, the dive supervisor decides “on the fly” to limit “in water” exposure, but also wants to run conservative decompression routine to compensate for the less than expected temperature. Therefore, the dive supervisor switches to the 33 metre surface O₂ table (see dive table 4, FIG. 9d), which will now give a schedule of:
a. 7 minutes at 9 m in water,
b. no more than a 7 minute interval on the surface for transfer to a hydrobaric chamber, and
c. 35 minutes in the chamber at an equivalent depth of 12 m while on Oxygen with one break breathing air for 5 minutes.

Therefore the diver's “in water” exposure is reduced by 34 minutes, reducing their time in the cold water. The dive plan still has a level of conservatism even though the dive plan has been changed mid-dive.

These are just four scenarios that the monitoring system selects and offers to the dive supervisor. The monitoring system provides these available scenarios to the dive supervisor, who can then select one that they deem appropriate. Once the revised dive plan is selected, the monitoring system will update the display to show the new decompression schedule based on the revised scenario/dive table. The system alarms will trigger based on the new scenario.

The monitoring system 13 will not allow a decompression schedule less than the safe minimum decompression schedules to be selected, but gives the dive supervisor flexibility to switch between dive tables in order to select the one that best suits the dive conditions. In the scenario above, the dive took some 3 minutes longer than planned and the water was colder than expected. However, with the scenario build, the topside monitoring system was able to present options to the dive supervisor “on the fly” that allowed the in water exposure to be limited to a safe level that effectively reduced the in water exposure by 34 minutes, but lengthened the total decompression to 55 minutes from the minimum of 41 minutes.

In the above example, the dive supervisor is offered options that are selected by the monitoring system 13. The dive supervisor has the ultimate control to decide on and select which decompression schedule should be followed. This decision will be based on their understanding of the situation, the parameters being received from the diver in questions, and the supervisor's experience.

In an alternative, the topside monitoring system 13 itself might automatically select an alternative decompression schedule using its software 112. It could do this based on the parameters being received from the diver. The topside monitoring system 13 would be programmed with rules to make the adjustment. For example, it might be programmed to select the most conservative alternative decompression schedule if any one of the dive time, bottom depth, temperature, available air change such that the current decompression schedule is not suitable, or is no longer possible.

The above dive plan revision was based on unexpected water temperature. Dive planning and revision, and diver management can be based on a large number of events that might occur, which are triggered by various dive parameters. For example, adjustment of a planned dive could occur for one or more or for the following reasons:
diver hypothermia or hyperthermia,
diver hyperventilation,
equipment fault,
unforeseen difficulties in the task,
sub sea currents,
weather/temperature changes,
diver biomedical/physiological events,
diver entanglement.

### Recordal of Dive Data

Traditionally, aspects of a dive are recorded in paper format by the dive supervisor. This record comprises risk assessment forms, diver forms, and dive plans. If dive plans change, these should also be recorded. These recorded details of the dive can then be analysed later, for example in the event of a dive accident. Traditionally, their have been issues with inaccurate or doctored dive information, or incomplete dive information.

The present invention records all the data received from the divers during a dive. The data can be recorded in a storage device 24 in the topside monitoring system computer 101, or in another separate local or remote storage location. Further, the system records all information, decisions and actions carried out by the dive supervisor and the monitoring system 10. This provides a full accurate record of the dive and all the decisions made. This allows the dive to be replayed for analysis at a later date. Further, the reported data can be encrypted, and the decryption keys obscured. This means that data can be viewed, however it cannot be altered. This step is important for integrity of data to allow accurate post analysis of any dives. It provides confidence that the information has not been doctored. In effect, the recorded data provides an audit trail that can be used in a variety of ways.

A further benefit of recording all the information, is that the information can be used to make decisions and provide restrictions on further dives. For example, where a particular diver has already completed a dive and the results of that dive were that they should not complete another dive within a set period, the monitoring system will not allow a dive supervisor to plan a dive or carry out a dive for that diver. Prior to allowing a dive, the monitoring system will review historical data relating to the diver in question. If a dive supervisor attempts to plan a dive for a registered diver or send them on a dive within the disallowed period, the system will provide an alarm. Further, the monitoring system 13 may prevent the dive supervisor from utilising the system for that diver—thus preventing them from initiating the dive. In another example, if a diver's medical data indicates that they should not attempt the planned dive, again an alarm may sound or the monitoring system 13 may lock that diver out from a dive.

The system 10 also allows for control and supervision of recreational dives, or other dives where there are a large number of divers 12. Supervision of recreational divers is very important, as many may lack experience and require significant guidance. However, presently there is no way to accurately know the parameters relating to their dive and therefore guide them. Tethered dives cannot be used for recreational diving, as the number of divers would prevent this due to the danger of entanglement. The present invention allows parameters relating to each diver 12 to be sent topside 13 via the acoustic modem 207. A dive supervisor can monitor key parameters of each of the divers, and then communicate with them via communication channels via the acoustic modem to guide them as to decompression, breathing and other aspects of their dive. This opens up the ability to control dives more carefully and improve diver safety.

### System/Software Administration Functions

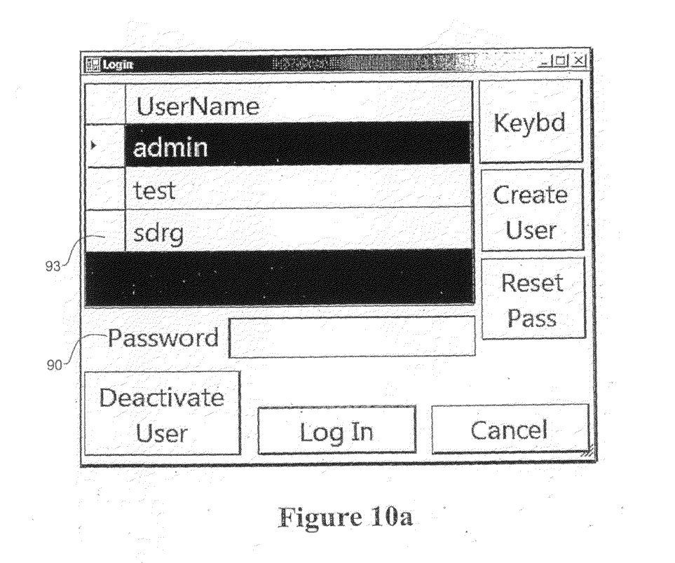

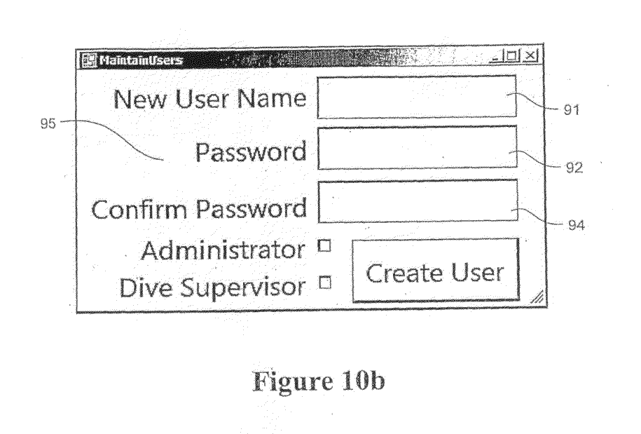

The administration module 23 provides administration functions. FIGS. 10a to 13 show the settings in the log in procedures for the invention. Referring to FIGS. 10a and 10b, each dive supervisor is preferably registered with a user name 91 and password 92 before using the a registration screen 95. Whenever the dive supervisor takes control of a dive they login using their details. The system will record all of the actions and data relating to dives for which they are the logged in as dive supervisor. This assists in analysing decisions made by particular dive supervisors at a later date. FIG. 10a shows a login page 90 listing registered dive supervisors 93 that can login. Referring to FIG. 10, a new dive supervisor login can be created by completing the required fields 91, 92, 94 on the registration screen 95.

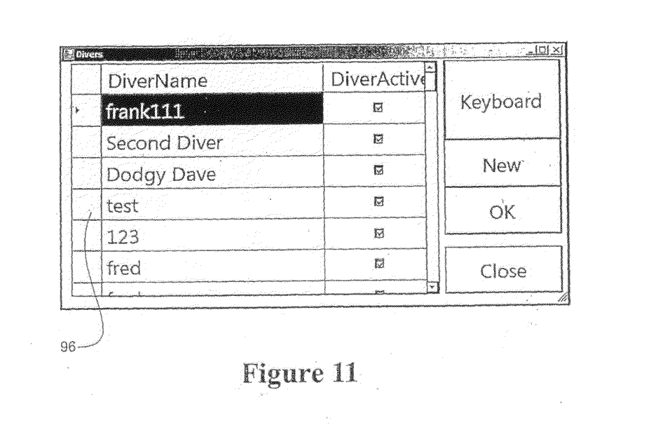

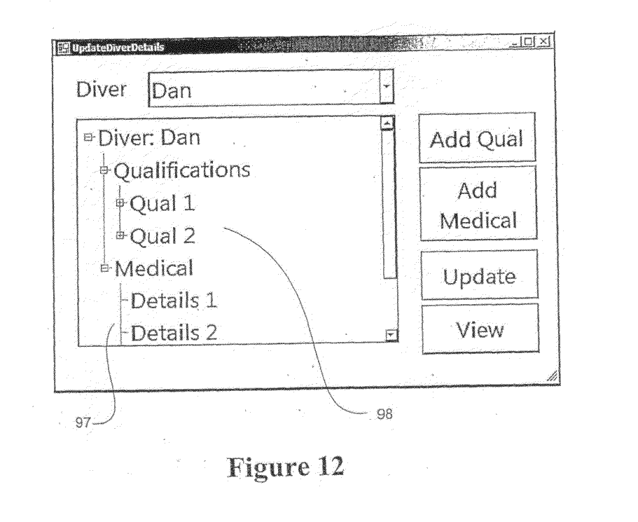

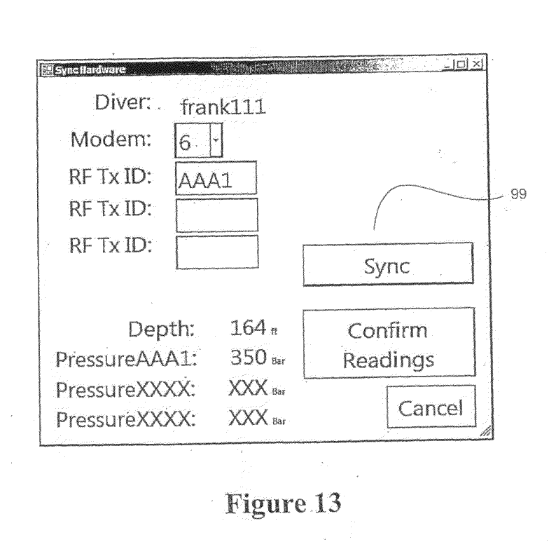

Referring to FIGS. 11 and 12, each diver who will perform or may perform a dive also is preferably registered. New divers can be added, or existing divers can have their details edited. FIG. 11 shows a screen 96 with registered divers, and gives options for creating new or editing old diver registrations. Referring to FIG. 12, upon clicking to register a new diver, a screen 97 is displayed where qualifications, medical and other details can be added to the diver registration profile 98. This information can be used in dive planning and also for generating alarms and for diver management over multiple dives. Decisions can be based on their medical details and qualifications. For a particular dive, a registered diver can be configured as shown in the screen 99 in FIG. 13 according to their hardware details.

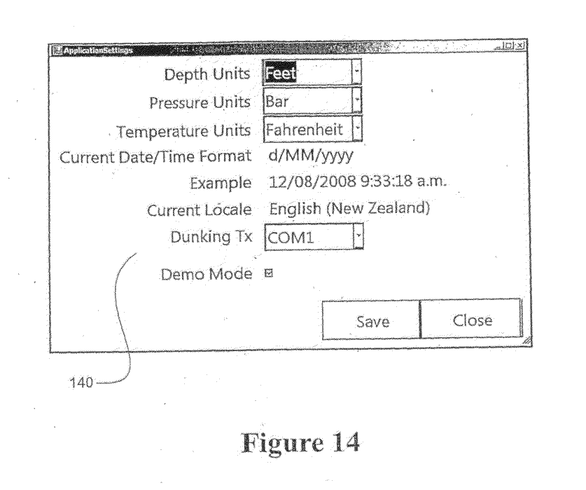

Referring to FIG. 14, various settings for the monitoring system can also be specified using the input screen 140. These comprise the units used by the dive monitoring system and various other formats.

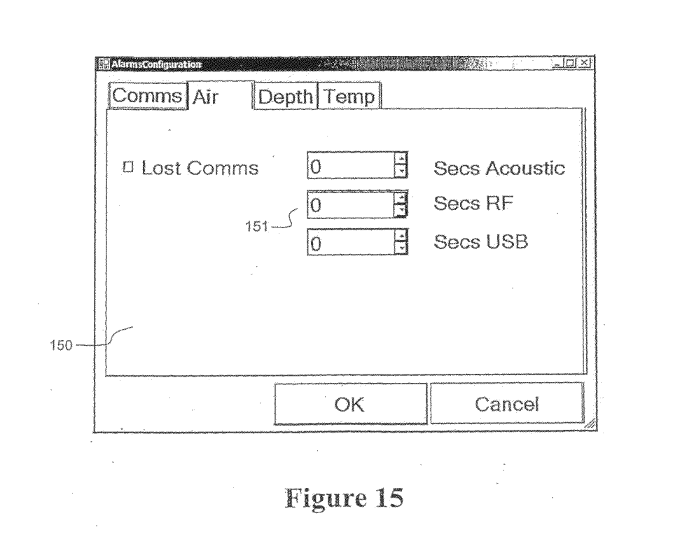

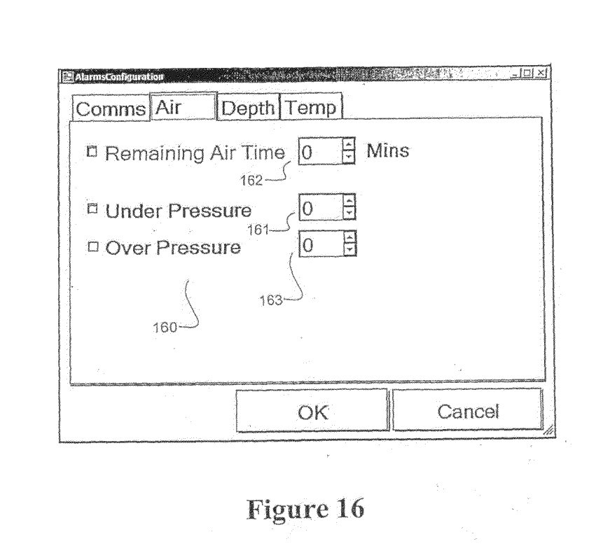

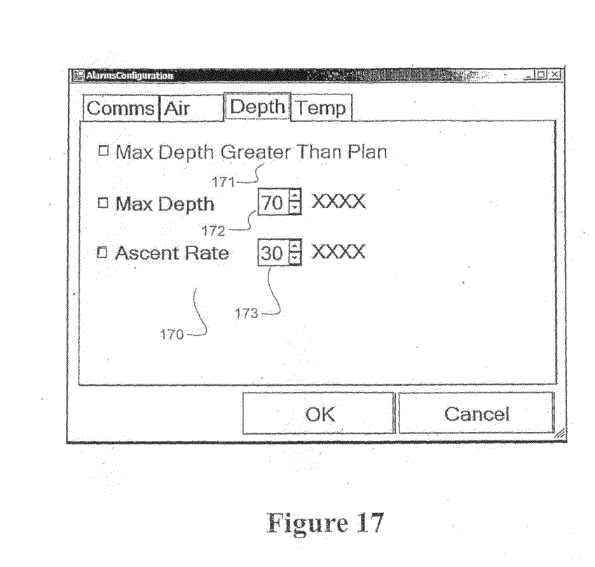

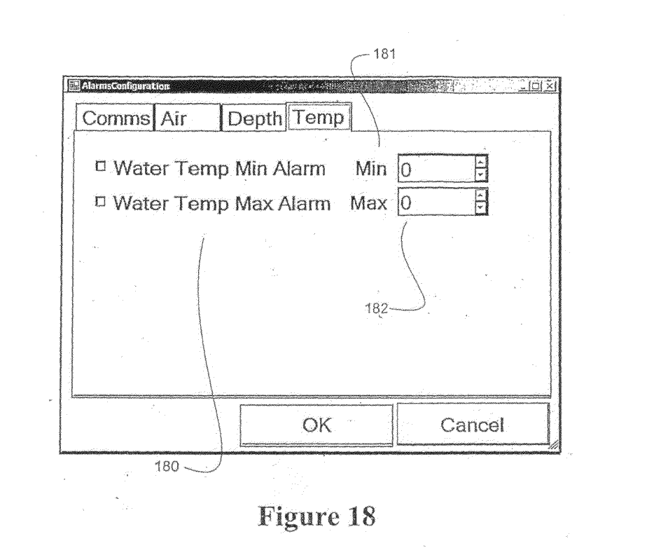

Alarms can be configured for various events, by completing the input fields shown in FIGS. 15 to 18. For example, as shown in the screen 150 in FIG. 15, if acoustic, RF or USB communications are lost for the time specified 151, an alarm will be provided. Referring to the screen 160 in FIG. 16, if air pressure drops below or goes above a specified threshold 161/163, or if remaining air time drops below a threshold 162, an alarm will be provided. Referring to the screen 170 in FIG. 17, if a maximum depth 172 is reached or if a depth greater than the specified plan depth 171 is reached or a maximum ascent rate is reached 173, then an alarm will be provided. Referring to the screen 180 in FIG. 18 if the water temperature reaches a max or minimum threshold 181/182, an alarm will be provided also. Other alarms could be specified, as will be appreciated by those skilled in the art.

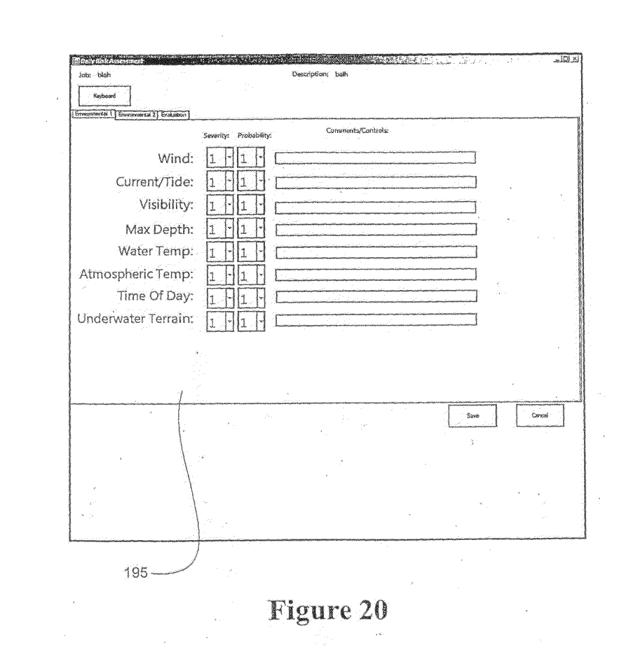

Referring to the screen 190 in FIG. 19, prior to a dive, various details 191 relating to the diver and the plan for the dive can be set by completing the fields 191. Further, weather and water conditions can be specified as shown in the screen 195 in FIG. 20.

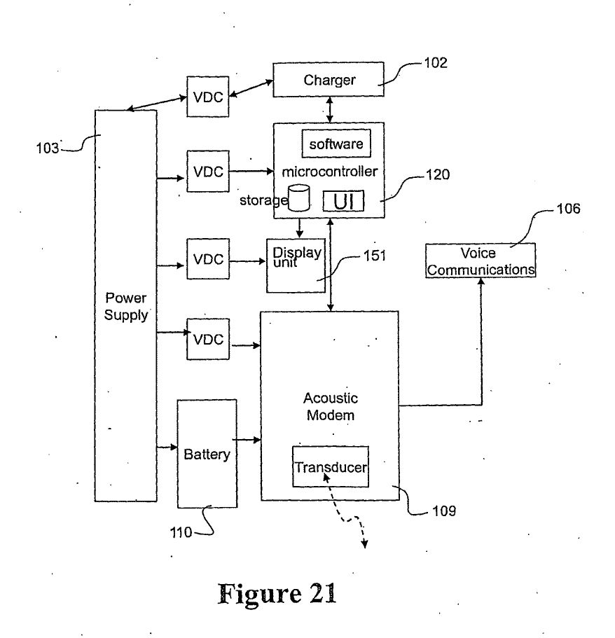

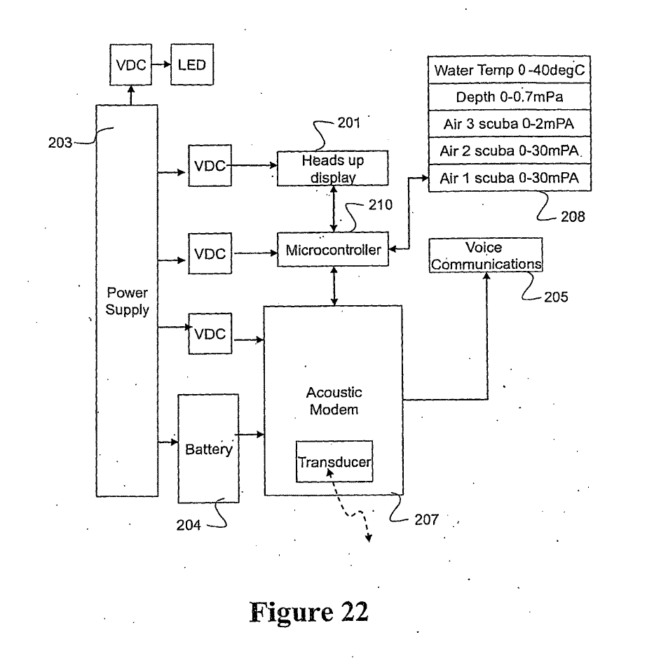

FIGS. 21 and 22 show alternative topside and diver-side systems respectively. These are the same as the systems described in FIGS. 2 and 3, except they do not provide tether communication.

FIG. 23 illustrates the range of the acoustic modem.

FIG. 24 illustrates an alternative display screen.

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

### APPENDIX A

The diver-side system and/or topside system could comprise sensors to obtain one or more of the following parameters:
surface supply breathing gas pressure at surface,
surface supply breathing gas composition at surface (oxygen concentration, nitrogen concentration, helium concentration, carbon dioxide concentration, carbon monoxide concentration and any other gas that is interest to the dive supervisor),
surface supply breathing gas pressure at diver,
surface supply breathing gas composition at diver (oxygen concentration, nitrogen concentration, helium concentration, carbon dioxide concentration, carbon monoxide concentration and any other gas that is interest to the dive supervisor or diver),
self-contained breathing gas supply pressure at diver for primary, secondary or tertiary use (compressed air for breathing, singular or mixed gas for breathing, gas for buoyancy compensation or driving of underwater tools),
self-contained breathing gas composition at diver (oxygen concentration, nitrogen concentration, helium concentration, carbon dioxide concentration, carbon monoxide concentration and any other gas that is interest to the dive supervisor or diver),
dive parameter sensors for altitude, depth, air temperature, water temperature, time.
photographic/video sensors for visual, enhanced visual, IR, UV,
water quality sensors for turbidity, suspended solids, salinity, biological, radioactive parameters,
environmental sensors for temperature, fluid movement and particle movement.
situational awareness sensors, sonar, radar, Doppler tracking of fluid and particles
positional sensors for absolute position and relative position
diver inhaled and exhaled fluid composition (oxygen gas concentration, carbon dioxide gas composition or any other gas parameter of interest to the diver and or diver supervisor)
diver biophysical condition sensors (ECG, EEG, respiration rate, core body and/or skin temperature)
dive supervisor electronic hardware sensors (battery condition, malfunction, self dialogistic)
diver electronic hardware sensors (battery condition, malfunction, self diagnostics)
diver microphone, speaker and microprocessor set for converting analogue sound to digital sound representation for data transmission.
dive supervisor microphone, speaker and microprocessor set for converting analogue sound to digital sound representation for data transmission.

### APPENDIX B

The monitoring system can be configured to provide warnings for one or more of the following:
surface supply breathing gas low pressure at surface
surface supply breathing gas low quality at surface
surface supply breathing gas low pressure at diver
surface supply breathing gas low quality at diver
primary and/or secondary self-contained breathing gas (or one of its components) low pressure at diver
primary and/or secondary self-contained breathing gas (or one of its components) low quality at diver
diving parameters:
   decompression commitment approaching (user defined, default 5 minutes),
   decompression commitment reached,
   partial pressure of oxygen in breathing gas mixture (user defined, default 1.4)
   accent to fast warning (user defined, default 18 m/min—could be graphical),
   remaining breathing gas warning on self contained supply (user defined default 15 minutes left),
   remaining breathing gas warning on secondary self contained supply (user defined—drop in pressure by 30 psi)
   maximum planned depth approaching (user defined—default 5 msw of planned depth)
   maximum planned depth reached (user defined—maximum 70 msw)
   minimum water temperature approaching (user defined—default 2° C. of set minimum)
   maximum water temperature approaching (user defined—default 2° C. of set maximum)
   minimum water temperature reached (user defined—default 10° C.)
   maximum water temperature approaching (user defined—default 30° C.)
dive environment
   low visibility for safe diver operations (user defined)
   presence of bacteriological activity (user defined)
   maximum level of radioactivity approaching (user defined)
   maximum level of radioactivity reached (user defined)
   current warning (user defined)
situational awareness warning
   approaching selected target (user defined—default range 5 m)
   selected target reached (user defined—default range 1 m)
   unknown target in vicinity of diver (user defined)
biophysical warning
   minimum core body and/or skin temperature approaching (user defined)
   minimum core body and/or skin temperature reached (user defined)
   maximum core body and/or skin temperature approaching (user defined)
   maximum core body and/or skin temperature reached (user defined)
   ECG anomaly (user defined)
   ECG rate high (user defined)
   ECG rate low (user defined)
   EEG anomaly (user defined)
   inhaled breathing gas quality low (user defined)
   exhaled breathing gas quality low (user defined)

1. A method of monitoring a diver comprising the steps of:
receiving data from at least one diver, the data being transmitted using an acoustic modem and specifying at least one parameter relating to the diver,
conveying the parameter and elapsed time of dive to a dive supervisor so that the dive supervisor can manage the dive, the parameter and elapsed time of dive forming diver information,
wherein the parameter relating to the diver comprises the depth of the diver.

2. A method according to claim 1 further comprising the step of determining a decompression schedule for the diver and conveying the decompression schedule.

3. A method according to claim 2 wherein the step of determining a decompression schedule comprises receiving input specifying one or more decompression schedules or dive tables, and determining a decompression schedule based on the received input.

4. A method according to claim 2 further comprising the steps of:
determining one or more alternative decompression schedules based on the conveyed diver information, the step being triggered by received or calculated diver information or by input from a dive supervisor, and
conveying the alternative decompression schedules.

5. (canceled)

6. (canceled)

7. (canceled)

8. A method according to claim 4 wherein the step of determining one or more alternative decompression schedules comprises selecting one or more dive tables from a set of dive tables, each dive table specifying one or more decompression schedules, wherein the selection is based on some or all of the diver information.

9. A method according to claim 8 wherein selecting one or more dive tables based on some or all of the diver information comprises the step of determining which of the set of dive tables specify decompression schedules that can still be adhered to by the diver based on diver information.

10. A method according to claim 1 further comprising one or more alarms for alerting a dive supervisor of actions that may be required, wherein the alarms are triggered by the relationship of:
diver information to the decompression schedule, and/or
diver information to specified values and/or states.

11. (canceled)

12. (canceled)

13. A method according to claim 1 further comprising the step of recording dive information related to the dive, wherein dive information comprises one or more of any dive related parameter, data, information or input received or generated prior to or throughout the dive.

14. A system for monitoring a diver comprising:
a topside monitoring system on a vessel comprising:
a receiver for receiving data from at least one diver, the data being transmitted using an acoustic modem and specifying information relating to the diver, wherein the information relating to the diver comprises the depth of diver,
a computer for processing the data, and
a user interface for conveying the information and the elapsed time of dive to a dive supervisor so that the dive supervisor can manage the dive.

15. A system according to claim 14 further comprising:
one or more sensors on the diver, the sensors for obtaining data relating to the depth of the diver, and
an acoustic modem on the diver for transmitting obtained data to the topside monitoring system

16. A system according to claim 14 wherein the computer is adapted to determine a decompression schedule for the diver and convey the decompression schedule via the user interface.

17. A system according to claim 16 wherein to determine a decompression schedule the computer is adapted to receive input specifying one or more decompression schedules or dive tables, and determine a decompression schedule based on the received input.

18. A system according to claim 17 wherein the computer is further adapted to
determine one or more alternative decompression schedules based on the conveyed diver information, the computer being triggered by received or calculated diver information or by input from a dive supervisor, and
the computer is adapted to convey the alternative decompression schedules via the user interface.

19. (canceled)

20. (canceled)

21. (canceled)

22. A system according to claim 18 wherein to determine one or more alternative decompression schedules the computer selects one or more dive tables from a set of dive tables, each dive table specifying one or more decompression schedules, wherein the selection is based on some or all of the diver information.

23. A system according to claim 22 wherein to select one or more dive tables based on some or all of the diver information the computer is adapted to determine which of the set of dive tables specify decompression schedules that can still be adhered to by the diver based on diver information.

24. A system according to claim 14 wherein the computer is configured with one or more alarms for alerting a dive supervisor of actions that may be required, wherein the alarms are triggered by the relationship of:
diver information to the decompression schedule, and/or
diver information to specified values and/or states.

25. A system according to claim 17 wherein receiving input to select a decompression schedule comprises:
receiving input indicating one or more dive tables, or decompression schedules,
receiving input indicating at least one parameter of the dive.

26. (canceled)

27. A system according to claim 14 wherein the computer is adapted to record dive information related to the dive, wherein dive information comprises one or more of any dive related parameter, data, information or input received or generated prior to or throughout the dive.

28. (canceled)

29. A system according to claim 15 comprising one or more further sensors on the diver, the sensors for obtaining data relating to one or more of the following parameters:
surface supply breathing gas pressure at surface,
surface supply breathing gas composition at surface (oxygen concentration, nitrogen concentration, helium concentration, carbon dioxide concentration, carbon monoxide concentration and any other gas that is interest to the dive supervisor),
surface supply breathing gas pressure at diver,
surface supply breathing gas composition at diver (oxygen concentration, nitrogen concentration, helium concentration, carbon dioxide concentration, carbon monoxide concentration and any other gas that is interest to the dive supervisor or diver),
self-contained breathing gas supply pressure at diver for primary, secondary or tertiary use (compressed air for breathing, singular or mixed gas for breathing, gas for buoyancy compensation or driving of underwater tools),
self-contained breathing gas composition at diver (oxygen concentration, nitrogen concentration, helium concentration, carbon dioxide concentration, carbon monoxide concentration and any other gas that is interest to the dive supervisor or diver),
dive parameter sensors for altitude, depth, air temperature, water temperature, time.
photographic/video sensors for visual, enhanced visual, IR, UV,
water quality sensors for turbidity, suspended solids, salinity, biological, radioactive parameters,
environmental sensors for temperature, fluid movement and particle movement.
situational awareness sensors, sonar, radar, Doppler tracking of fluid and particles
positional sensors for absolute position and relative position
diver inhaled and exhaled fluid composition (oxygen gas concentration, carbon dioxide gas composition or any other gas parameter of interest to the diver and or diver supervisor)
diver biophysical condition sensors (ECG, EEG, respiration rate, core body and/or skin temperature)
dive supervisor electronic hardware sensors (battery condition, malfunction, self dialogistic)
diver electronic hardware sensors (battery condition, malfunction, self diagnostics)
diver microphone, speaker and microprocessor set for converting analogue sound to digital sound representation for data transmission.
dive supervisor microphone, speaker and microprocessor set for converting analogue sound to digital sound representation for data transmission.

30. A method according to claim 1 wherein the diver information can further comprise one or more of the following parameters:
surface supply breathing gas pressure at surface,
surface supply breathing gas composition at surface (oxygen concentration, nitrogen concentration, helium concentration, carbon dioxide concentration, carbon monoxide concentration and any other gas that is interest to the dive supervisor),
surface supply breathing gas pressure at diver,
surface supply breathing gas composition at diver (oxygen concentration, nitrogen concentration, helium concentration, carbon dioxide concentration, carbon monoxide concentration and any other gas that is interest to the dive supervisor or diver),
self-contained breathing gas supply pressure at diver for primary, secondary or tertiary use (compressed air for breathing, singular or mixed gas for breathing, gas for buoyancy compensation or driving of underwater tools),
self-contained breathing gas composition at diver (oxygen concentration, nitrogen concentration, helium concentration, carbon dioxide concentration, carbon monoxide concentration and any other gas that is interest to the dive supervisor or diver),
dive parameter sensors for altitude, depth, air temperature, water temperature, time.
photographic/video sensors for visual, enhanced visual, IR, UV,
water quality sensors for turbidity, suspended solids, salinity, biological, radioactive parameters,
environmental sensors for temperature, fluid movement and particle movement.
situational awareness sensors, sonar, radar, Doppler tracking of fluid and particles
positional sensors for absolute position and relative position
diver inhaled and exhaled fluid composition (oxygen gas concentration, carbon dioxide gas composition or any other gas parameter of interest to the diver and or diver supervisor)
diver biophysical condition sensors (ECG, EEG, respiration rate, core body and/or skin temperature)
dive supervisor electronic hardware sensors (battery condition, malfunction, self dialogistic)
diver electronic hardware sensors (battery condition, malfunction, self diagnostics)
diver microphone, speaker and microprocessor set for converting analogue sound to digital sound representation for data transmission.
dive supervisor microphone, speaker and microprocessor set for converting analogue sound to digital sound representation for data transmission.

31. A method according to claim 10 wherein one or more alarms can be triggered based on one or more of:
surface supply breathing gas low pressure at surface
surface supply breathing gas low quality at surface
surface supply breathing gas low pressure at diver
surface supply breathing gas low quality at diver
primary and/or secondary self-contained breathing gas (or one of its components) low pressure at diver
primary and/or secondary self-contained breathing gas (or one of its components) low quality at diver
diving parameters:
decompression commitment approaching (user defined, default 5 minutes),
decompression commitment reached,
partial pressure of oxygen in breathing gas mixture (user defined, default 1.4)
accent to fast warning (user defined, default 18 m/min—could be graphical),
remaining breathing gas warning on self contained supply (user defined default 15 minutes left),
remaining breathing gas warning on secondary self contained supply (user defined—drop in pressure by 30 psi)
maximum planned depth approaching (user defined—default 5 msw of planned depth)
maximum planned depth reached (user defined—maximum 70 msw)
minimum water temperature approaching (user defined—default 2° C. of set minimum)
maximum water temperature approaching (user defined—default 2° C. of set maximum)
minimum water temperature reached (user defined—default 10° C.)
maximum water temperature approaching (user defined—default 30° C.)
dive environment
low visibility for safe diver operations (user defined)
presence of bacteriological activity (user defined)
maximum level of radioactivity approaching (user defined)
maximum level of radioactivity reached (user defined)
current warning (user defined)
situational awareness warning
approaching selected target (user defined—default range 5 m)
selected target reached (user defined—default range 1 m)
unknown target in vicinity of diver (user defined)
biophysical warning
minimum core body and/or skin temperature approaching (user defined)
minimum core body and/or skin temperature reached (user defined)
maximum core body and/or skin temperature approaching (user defined)
maximum core body and/or skin temperature reached (user defined)
ECG anomaly (user defined)
ECG rate high (user defined)
ECG rate low (user defined)
EEG anomaly (user defined)
inhaled breathing gas quality low (user defined)
exhaled breathing gas quality low (user defined)

32. (canceled)

33. (canceled)

34. A system according to claim 24 wherein one or more alarms can be triggered based on one or more of:
surface supply breathing gas low pressure at surface
surface supply breathing gas low quality at surface
surface supply breathing gas low pressure at diver
surface supply breathing gas low quality at diver
primary and/or secondary self-contained breathing gas (or one of its components) low pressure at diver
primary and/or secondary self-contained breathing gas (or one of its components) low quality at diver
diving parameters:
decompression commitment approaching (user defined, default 5 minutes),
decompression commitment reached,
partial pressure of oxygen in breathing gas mixture (user defined, default 1.4)
accent to fast warning (user defined, default 18 m/min—could be graphical),
remaining breathing gas warning on self contained supply (user defined default 15 minutes left),
remaining breathing gas warning on secondary self contained supply (user defined—drop in pressure by 30 psi)
maximum planned depth approaching (user defined—default 5 msw of planned depth)
maximum planned depth reached (user defined—maximum 70 msw)
minimum water temperature approaching (user defined—default 2° C. of set minimum)
maximum water temperature approaching (user defined—default 2° C. of set maximum)
minimum water temperature reached (user defined—default 10° C.)
maximum water temperature approaching (user defined—default 30° C.)
dive environment
low visibility for safe diver operations (user defined)
presence of bacteriological activity (user defined)
maximum level of radioactivity approaching (user defined)
maximum level of radioactivity reached (user defined)
current warning (user defined)
situational awareness warning
approaching selected target (user defined—default range 5 m)
selected target reached (user defined—default range 1 m)
unknown target in vicinity of diver (user defined)
biophysical warning
minimum core body and/or skin temperature approaching (user defined)
minimum core body and/or skin temperature reached (user defined)
maximum core body and/or skin temperature approaching (user defined)
maximum core body and/or skin temperature reached (user defined)
ECG anomaly (user defined)
ECG rate high (user defined)
ECG rate low (user defined)
EEG anomaly (user defined)
inhaled breathing gas quality low (user defined)
exhaled breathing gas quality low (user defined)

35. A method of monitoring a diver comprising the steps of:
receiving data at a topside computer system from at least one diver during a dive, the data being transmitted using an acoustic modem and specifying a plurality of parameters relating to the diver and/or ambient conditions and comprising at least the depth of the diver,
conveying the parameters and elapsed time of dive to a dive supervisor on a user interface of the computer system so that the dive supervisor can manage the dive, the parameter and elapsed time of dive forming diver information,
determining whether a dive decompression schedule can be complied with by the diver based on the diver information, and if not, selecting a further decompression schedule from a set of dive tables that can be complied with by the diver based on the diver information, and
conveying the further decompression schedule to the dive supervisor on the user interface.

36. A system for monitoring a diver comprising:
a topside monitoring system on a vessel comprising:
a receiver for receiving data from at least one diver, the data being transmitted using an acoustic modem and specifying information relating to the diver, wherein the information relating to the diver comprises at least the depth of diver,
a computer for processing the data, and
a user interface for:
conveying the information and the elapsed time of dive to a dive supervisor so that the dive supervisor can manage the dive, the information and elapsed time of dive forming diver information,
conveying compressions schedules,
wherein
the computer is adapted to determine whether a dive compression schedule can be complied with by the diver based on the diver information, and if not, select a further decompression schedule from a set of dive tables that can be complied with by the diver based on the diver information.
